(19) United States
(12) Patent Application Publication
(10) Pub. No.: US 2013/0018172 A1
(43) Pub. Date: Jan. 17, 2013

(54) Metal Abstraction Peptide and Uses Thereof

(71) Applicants: Jennifer Ann Stowell Laurence, Lawrence, KS (US); Mary Elizabeth Krause, Lawrence, KS (US); Timothy A. Jackson, Lawrence, KS (US); George Laurence, Lawrence, KS (US)

(72) Inventors: Jennifer Ann Stowell Laurence, Lawrence, KS (US); Mary Elizabeth Krause, Lawrence, KS (US); Timothy A. Jackson, Lawrence, KS (US); George Laurence, Lawrence, KS (US)

(73) Assignees: Echogen, Inc., Lenexa, KS (US); University of Kansas, Lawrence, KS (US)

(21) Appl. No.: 13/350,247

(22) Filed: Jan. 13, 2012

Related U.S. Application Data
(60) Provisional application No. 61/432,231, filed on Jan. 13, 2011.
(60) Provisional application No. 61/493,557, filed on Jun. 6, 2011.

(51) Int. Cl. C07K 4/00; C07K 19/00; C07K 1/22; C07K 14/00; C07K 1/113; C07K 1/107

(52) U.S. Cl. 530/331; 530/345; 530/300; 530/402; 530/413; 530/350; 530/409

(57) ABSTRACT

Compositions comprising certain tripeptides which are capable of binding a metal in a square planar orientation, a square pyramidal orientation, or both, are disclosed in processes for modulating tagged peptides or proteins. Such compositions and processes may be used for site-specific chiral inversion of amino acids, site-specific peptide cleavage, and protein purification, among other uses.

### CROSS-REFERENCE

This application claims the benefit of U.S. Provisional Application No. 61/432,231, filed Jan. 13, 2011, and U.S. Provisional Application No. 61/493,557, filed Jun. 6, 2011, each of which are incorporated herein by reference.

### BACKGROUND OF THE INVENTION

Tags that can be encoded in the genetic material of an organism for recombinant expression of proteins have been utilized for purification and identification of protein products. The advantage of a peptide tag is that the tag is covalently attached to the protein of interest without the need for additional chemical steps to label the protein. One example of such a tag is a poly His-tag, which provides a means of isolating the tagged protein from whole cells using immobilized metal affinity chromatography (IMAC). Another example of a peptide tag can be found in U.S. Pat. No. 7,208,138 by Haroon, which refers to a peptide having the sequence NXEQVSP (SEQ ID NO: 1). An additional example of a peptide tag can be found in U.S. Patent Publication 2004/0018974 by Arbogast, which refers to a tag sequence that appears to be an entire protein. Other peptide-based tags have been developed to allow for detecting a tagged protein in cell culture assays or cell lysates using antibodies that recognize the peptide tag.

While these technologies might be useful in in situ or in vitro assays, they generally are not applicable to in vivo analysis. Moreover, such peptide tags have limited or no functionality outside of protein purification or identification. Thus, there remains a need for peptide tags with metal-binding properties providing increased functionality, such as site-specific modulation of the tagged protein.

### SUMMARY OF THE INVENTION

In one aspect, a method is disclosed of performing a site-specific modulation of a tripeptide comprising complexing a metal ion with a tripeptide having the sequence LXLC₁LC₂ to form a metal ion-DXLC₁DC₂ complex, wherein X is any amino acid such that the tripeptide and metal ion form a complex having a square planar orientation or square pyramidal orientation or both, and wherein C₁ and C₂ are the same or different; and wherein C₁ is chosen from a cysteine and a cysteine-like nonnatural amino acid; and wherein C₂ is chosen from a cysteine, a cysteine-like nonnatural amino acid, a histidine, and a histidine-like nonnatural amino acid.

In various embodiments, the method is a method of performing a site-specific chiral inversion. Preferably, the metal ion selected from the group consisting of nickel and palladium.

In one aspect, a method is disclosed of performing a site-specific modulation of a tripeptide comprising complexing a metal ion with a tripeptide having the sequence GLC₁LC₂ to form a metal ion-GLC₁DC₂ complex, wherein the L prefix represents the L-enantomer, the D prefix represents the D-enantomer, G is glycine such that the tripeptide and metal ion form a complex having a square planar orientation or square pyramidal orientation or both, and wherein C₁ and C₂ are the same or different; and wherein C₁ is chosen from a cysteine and a cysteine-like nonnatural amino acid; and wherein C₂ is chosen from a cysteine, a cysteine-like nonnatural amino acid, a histidine, and a histidine-like nonnatural amino acid.

In various embodiments, the method is a method of performing a site-specific chiral inversion. Preferably, the metal ion is selected from the group consisting of nickel and palladium.

In one aspect, a method is disclosed of providing anti-oxidant properties to a composition comprising providing a plurality of amino acids having the sequence LXLC₁LC₂ and a metal ion and allowing the site-specific chiral inversion of the sequence LXLC₁LC₂; wherein X is any amino acid such that the tripeptide and metal ion form a complex having a square planar orientation or square pyramidal orientation or both, and wherein C₁ and C₂ are the same or different; and wherein C₁ is chosen from a cysteine and a cysteine-like nonnatural amino acid; and wherein C₂ is chosen from a cysteine, a cysteine-like nonnatural amino acid, a histidine, and a histidine-like nonnatural amino acid. An anti-oxidant composition is disclosed comprising a metal ion complexed with a tripeptide having the sequence LXLC₁LC₂ to form a metal ion-DXLC₁DC₂ complex; wherein X is any amino acid such that the tripeptide and metal ion form a complex having a square planar orientation or square pyramidal orientation or both, and wherein C₁ and C₂ are the same or different; and wherein C₁ is chosen from a cysteine and a cysteine-like nonnatural amino acid; and wherein C₂ is chosen from a cysteine, a cysteine-like nonnatural amino acid, a histidine, and a histidine-like nonnatural amino acid.

In one aspect, a method is disclosed for performing a site-specific cleavage further comprising: providing a partial chelator with a metal capable of reacting with the sequence XC₁C₂; and allowing cleavage of the peptide within or at a terminal portion of the sequence XC₁C₂. Preferably, the metal is copper or iron. In various embodiments, the method further comprises an electron transfer component. For example, the electron transfer component may be selected from O₂ or a species donating an electron.

In various embodiments of the method of site-specific cleavage, the peptide further comprises at least one selected from the group consisting of: an affinity tag, a peptide, and a protein. In embodiments further comprises an affinity tag, the method may further comprise immobilizing the peptide on a substrate for the affinity tag; and releasing a portion of the peptide from the substrate. Preferably, the method includes recovering a portion of the peptide.

In one aspect, a composition is disclosed comprising a nucleic acid sequence encoding the amino acid sequence of XC₁C₂, wherein X is any amino acid, C₁ and C₂ are the same or different, C₁ is chosen from a cysteine and a cysteine-like nonnatural amino acid, and C₂ is chosen from a cysteine, a cysteine-like nonnatural amino acid, a histidine, and a histidine-like nonnatural amino acid. Preferably, the composition includes a nucleotide sequence for expression. In various embodiments, a kit is disclosed comprising a peptide having a sequence comprising XC₁C₂, wherein X is any amino acid, C₁ and C₂ are the same or different, C₁ is chosen from a cysteine and a cysteine-like nonnatural amino acid, and C₂ is chosen from a cysteine, a cysteine-like nonnatural amino acid, a histidine, and a histidine-like nonnatural amino acid; and a partial chelator with a metal capable of reacting with the sequence XC₁C₂.

In one aspect, a method is disclosed of protein purification comprising: a) binding a first composition comprising a target protein associated with a linker and an affinity tag to an affinity material comprising a ligand capable of capturing the first composition; b) providing a metal-containing cleavage agent capable of detaching the target protein from the first composition such that the detached target protein is devoid of any supplementary amino acids originating from the linker or the affinity tag; and c) separating the target protein from the first composition.

In various embodiments, the cleavage agent comprises a metal ion, such as copper, including Cu(II). Preferably, the cleavage agent partially chelates the metal ion.

According to various embodiments of the above, the linker comprises a peptide sequence comprising a tripeptide with the sequence XC₁C₂, wherein X is any amino acid and wherein C₁ is chosen from a cysteine and a cysteine-like nonnatural amino acid, and C₂ is chosen from a cysteine, a cysteine-like nonnatural amino acid, a histidine, and a histidine-like nonnatural amino acid.

Likewise, the affinity tag comprises a tag from the group consisting of maltose binding protein, gluotathione-S-transferase, poly(His), biotin ligase tags, Strep, HaloTag, cellulose binding domain, gluotathione transferase, and glycan.

In various embodiments, the ligand is associated with a substrate. Likewise, the affinity tag in the first composition binds the ligand in the affinity material irreversibly. Preferably, the linker in the first composition binds to and removes the metal ion from the cleavage agent.

In various embodiments, the method further comprises preparing the target protein for therapeutic administration.

In one aspect, a method is disclosed of protein purification comprising: a) providing a first composition comprising a target protein associated with a linker and an affinity tag to an affinity material comprising a ligand capable of capturing the first composition; b) allowing said first composition and said affinity material to bind; c) providing a metal-containing cleavage agent capable of detaching the target protein from the first composition such that the detached target protein is devoid of any supplementary amino acids originating from the linker or the affinity tag; and d) separating the target protein from the first composition; wherein the affinity material is discarded after a single use.

In one aspect, a composition is disclosed for purifying a protein comprising; a) an affinity tag, wherein the affinity tag is capable of binding to a ligand; and b) a linker associated with the affinity tag, wherein the linker is covalently attached to a target protein; wherein the entirety of the affinity tag and the linker are capable of being detached from the target protein by the addition of a cleavage agent comprising a metal ion.

In various embodiments, a composition is disclosed for purifying a protein comprising; a) an affinity domain, wherein the affinity domain is capable of binding to a ligand; and b) a linker domain associated with the affinity domain, wherein the linker is covalently attached to a target protein; wherein the affinity domain and the linker domain are capable of being detached from the target protein such that the detached target protein is devoid of any supplementary amino acids originating from the linker or the affinity tag. Preferably, the capability to detach is dependent on the addition of a cleavage agent comprising a metal ion.

In various embodiments, the linker comprises a peptide sequence comprising a tripeptide with the sequence XC₁C₂, wherein X is any amino acid and wherein C₁ is chosen from a cysteine and a cysteine-like nonnatural amino acid, and C₂ is chosen from a cysteine, a cysteine-like nonnatural amino acid, a histidine, and a histidine-like nonnatural amino acid. Preferably, the cleavage agent comprises copper, such as Cu(II).

In various embodiments, the metal ion is partially chelated in the cleavage agent.

Preferably, the affinity tag comprises a tag from the group consisting of maltose binding protein, gluotathione-S-transferase, poly(His), biotin ligase tags, Strep, HaloTag, cellulose binding domain, gluotathione transferase, and glycan. In various embodiments, the ligand is associated with a substrate. Preferably, the affinity tag is capable of binding the ligand irreversibly. In various embodiments, the linker is capable of capturing the metal ion in said cleavage agent irreversibly.

In one aspect, a protein is disclosed wherein the protein is purified according to the methods disclosed herein. In various embodiments, the protein has no residual tags or remnants of the purification process. In various embodiments, a composition comprising the purified protein where the tag has been cleaved also contains a residual amount of tagged protein. For example, a composition is disclosed comprising a purified protein and further comprising the protein coupled with (i) an affinity tag, (ii) a linker, or (iii) an affinity tag and a linker. Preferably, the linker comprises a peptide sequence comprising a tripeptide with the sequence XC₁C₂, wherein X is any amino acid and wherein C₁ is chosen from a cysteine and a cysteine-like nonnatural amino acid, and C₂ is chosen from a cysteine, a cysteine-like nonnatural amino acid, a histidine, and a histidine-like nonnatural amino acid. In various embodiments, the protein is contained in a composition that also contains the cleavage agent described herein. In one example, the cleavage agent comprises a partial chelator and a metal, such as copper. The amounts of tagged protein or cleavage agent may be less than 1% by weight, such as 0.5%, 0.1%, or less, compared to the weight of purified protein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-E shows various arrangements for alignment of a metal activated cleavage sequence (*) with other sequences.

FIG. 2 shows SEQ ID NO:2, a nucleotide sequence of a cellulose binding domain; SEQ ID NO:3, the complement of SEQ ID NO:2, and SEQ ID NO:4, the deduced amino acid sequence. See U.S. Pat. No. 5,496,934, which is incorporated by reference.

FIG. 3 shows a consensus sequence for the cellulose binding domain. Amino acid sequence alignments of the cellulose-binding domains (CDBs) of C. fimi cellulases (CenA (SEQ ID NO:5) and Cex (SEQ ID NO:6)), and the putative binding domains of MbCelA (an endoglucanase from Microbispora bispora) (SEQ ID NO: 7), ClfX (part of the translated open reading frame of Cellulomonas flavigena gene fragment) (SEQ ID NO: 8), Pfegl (an endoglucanase from Pseudomonas fluorescens var. cellulosa) (SEQ ID NO: 9), PfxynA (a xylanase from P. fluorescens var. cellulosa) (SEQ ID NO:10) and BfeglI (an endoglucanase from Butyivibria fibrisolvens) (SEQ ID NO: 11). Amino acid residues are indicated in single letter code. Bold upper case letters indicate homology with the CenA sequence; plain upper case letters indicate homologies occurring only within the other six sequences; lower case letters indicate absence of homology. * indicates the amino terminus of the mature enzyme. * * * indicates a carboxyl terminus deduced from occurrence of stop codons in corresponding DNA sequences. - indicates a gap left to improve the alignment. Numbers refer to residues at the start and end of respective lines: CenA, Cex, and PfxynA residues are numbered from the start of the mature proteins; MbCelA, Pfend and Bfendl are numbered from the start of the unprocessed polypeptides since the sites of leader peptide processing have not been determined; ClfX is numbered from the start of the C. flavigena gene fragment open reading frame. See U.S. Pat. No. 5,340,731, which is incorporated by reference.

FIG. 4 shows SEQ ID NO: 12 and SEQ ID NO: 13, which provide a consensus cellulose binding domain and also an RGD tripeptide. See U.S. Pat. No. 6,407,208.

FIG. 5 shows SEQ ID NO: 14, which provides a consensus cellulose binding domain. See U.S. Pat. No. 6,407,208.

FIG. 6 shows SEQ ID NO: 15 and SEQ ID NO: 16, which provide a consensus cellulose binding domain as an oligonucleotide sequence and an amino acid sequence. See U.S. Pat. No. 6,407,208.

FIG. 7 shows SEQ ID NO: 17, which provides an alternate consensus cellulose binding domain. See U.S. Pat. No. 6,407,208.

FIG. 8 shows SEQ ID NO: 18, which provides a chimeric protein comprising a cellulose-binding domain. See U.S. Pat. No. 6,407,208.

FIG. 9 shows SEQ ID NO: 19, which shows an example of the sequence of a cellulase which can be joined to a cellulose-binding domain. See U.S. Pat. No. 6,407,208.

FIG. 10 shows SEQ ID NOs: 20-22, which provides a modified DNA sequence encoding a cellulose binding domain and an RGD tripeptide. See U.S. Pat. No. 6,407,208.

FIG. 11 shows a sequence alignment of the C-terminal portion of human PRL enzymes, including PR1-1, PRL-2, and PRL-3. H166 of PRL-1 is part of the NGH motif shown underlined. Residues C170 and C171 of PRL-1 are part of the CaaX motif shown in italics. The NCC motif is bold.

FIG. 12A shows electronic absorption spectra of PRL-1 variants purified in the presence of Ni²⁺ ion. Three (two) spectra for each protein variant were recorded from 800 to 200 nm and averaged. Protein concentration for all variants is 5 mg/mL to within 10% based on A280 comparison. Shown are the spectra from 700 to 300 nm. Visible features are absorbance maxima at 318 and 421 nm. Inset. The spectra from 600 to 400 nm are shown at smaller scale for visualization of absorption maxima at 421 and 526 nm. Curves are as follows: PRL-1-WT, black (top); PRL-1-H166A, (next to top); PRL-1-C170S-C171S, (bottom); PRL-1-C1705, (next to bottom); PRL-1-C171S, (middle). FIG. 12B shows resolved spectrum of PRL-1 WT. Resolution of the UV-Vis spectrum of Ni-purified PRL-1 WT protein between 300 and 700 nm fits the data using 5 curves. The 318 nm shoulder is resolved into peaks at 306, 325, and 372 nm. The sum of the resolved absorbance peaks (gray dashed line) and the raw data (solid black line) are both shown. The peaks at 421 and 526 nm are also present.

FIG. 13A shows ESI-MS of the nickel-bound tripeptide acquired at pH 10, where the mass indicates that nickel is bound to the peptide. FIG. 13B shows ESI-MS spectrum after dropping the pH of the sample to 5, showing that nickel has been released from the peptide.

FIG. 14 shows Cu-NCC magnetic resonance image (MRI).

FIG. 15 shows Structure 1—a GGH-like metal complex NGH, with 4N coordination. Structure 2 depicts a GGH-like metal coordination by a NCC peptide, whereas Structure 3 depicts the unique, high-affinity binding arrangement of NCC, involving 2N:2S coordination.

FIG. 16 shows the CD profile of species at various pH. See Example 8.

FIG. 17 shows the effect of argon sparging vs air for Cu-NCC species.

FIG. 18 shows the effect of copper with 8mer peptides, transmetallated with IMAC resin, at a pH of 7.4 in 50 mM phosphate buffer. The results show that the sequence and the incorporation conditions such as pH and buffer may be used to tune the kinetics of the cleavage reaction.

FIG. 19 showsthe effect of copper with 8mer peptides, transmetallated with Cu-IMAC resin at higher pH (around 8.5) in 2:1 phosphate/borate.

FIG. 20 shows that in different buffer systems, Ni-NCC has different CD spectral features.

FIG. 21 shows an aging progression of Ni-NCC in phosphate.

FIG. 22A shows electronic absorption spectra of freshly prepared and aged Ni-NCC in 50 mM phosphate buffer at pH 7.4. FIG. 22B shows CD spectra of the same samples. Individual Gaussian curves ( . . . ) and their sums ( - - - ) obtained from fits of the CD data are displayed.

FIG. 23 shows CD spectra of Ni-NCC aged in phosphate buffer vs. DLD-Ni-NCC.

FIG. 24 shows CD spectra depicting the aging progression of various chiral versions of the Ni-NCC complex.

FIG. 25 shows energy minimized structures of LLL-, DLL-, and DLD-Ni-NCC. The only hydrogen atoms shown are those attached to the Calpha of the residues. Views perpendicular to the square plane (a) and in the plane (b) are shown.

FIG. 26 shows CD spectra of aging Ni-NCC in pH 7.4 potassium phosphate buffer exposed to air (top) and purged with Ar (bottom). Darker spectra were obtained at the time the absorption spectra were at maximum intensity, indicating maximum LLL-Ni-NCC complex formation (t=10 minutes for O2 exposed and t=300 minutes for O2-free), lighter spectra were collected at t=590 minutes, and intermediate spectra obtained every ten minutes between these two times are represented as black dotted lines.

FIG. 27 shows CD spectra of Ni-NCC generated O2-free and incubated 120 minutes to form LLL-Ni-NCC complex, then injected with O2 (top), Ni-NCC generated O2-exposed (middle), and Ni-NCC generated O2-free (bottom). Darker spectra were obtained at t=10 minutes for the O2-exposed sample and t=130 minutes for the sample prepared O2-free and the sample formed O2-free, then injected with O2. The lighter spectra were collected after t=600 minutes for all three samples. Intermediate spectra obtained every ten minutes between these two times are represented as black dotted lines.

FIG. 28 shows a plot of the change in intensity of the CD signal at 19 200 cm⁻¹ as a function of exposure to oxygen in air.

FIG. 29 shows a plot of the change in intensity of the CD signal at 19 200 cm⁻¹ verses the amount of SOD activity as a function of exposure to oxygen in air.

### DESCRIPTION

The present disclosure generally relates to metal abstraction peptide tags (MAP tags) and to methods of preparing and using such tags for site-specific modulation. In particular, compositions and methods according to the invention relate to site-specific chiral inversion of amino acids or site-specific cleavage of peptides.

### DEFINITIONS

As used herein, the abbreviations for the natural L-enantiomeric amino acids are conventional and are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Typically, Xaa may indicate any amino acid. However, in some embodiments, X may be asparagine (N), glutamine (Q), histidine (H), lysine (K), or arginine (R).

When an amino acid sequence is represented as a series of three-letter or one-letter amino acid abbreviations, it will be understood that the left-hand direction is the amino terminal direction and the right-hand direction is the carboxy terminal direction, in accordance with standard usage and convention.

The term “metal” as used herein refers to metals in elemental form, metal atoms, and metal ions interchangeably.

The terms “purification”, “separation”, “extraction” and “isolation” are used interchangeably to refer to the process of separating a target polypeptide from other components in a polypeptide-containing sample.

“Operably linked” refers to a linkage in which the regulatory DNA sequences and the DNA sequence to be expressed are connected in such a way as to permit transcription and ultimately translation.

The term “host cell” refers to those cells capable of growth in culture and capable of hosting nucleic acids encoding the protein-based constructs described herein.

The term “X % sequence similarity” is not intended to be limited to sequences having a X % sequence similarity over their entire length, but rather includes sequence similarity over a portion of the length of the polynucleotide or polypeptide.

The term “CBD functional derivative” as used herein refers to any fragment, variant, analog, or chemical derivative of the CBD protein amino acid sequence described herein which retains the capability of binding to cellulose with high affinity and in a reversible manner and is preferably between about 2 and about 160 amino acids in length, more preferably between about 25 and about 125 amino acids in length and most preferably between about 50 and about 100 amino acids in length.

Metal Abstraction Peptide (MAP) Tag

The present disclosure generally relates to tripeptide motifs and methods of using such motifs. These peptides have the ability to bind to metals, which makes them useful for a variety of applications. In particular, the tripeptides of the present disclosure have applications in site-specific modulation of peptides or proteins to which they are linked. End uses of the modulated proteins may include imaging, research, therapeutics, pharmaceuticals, chemotherapy, chelation therapy, and metal sequestering.

The present disclosure provides a tripeptide having the sequence XC₁C₂; wherein X is any natural or non-natural amino acid or amino acid analog such that XC₁C₂ is capable of binding a metal in a square planar orientation or square pyramidal orientation or both; and wherein C₁ and C₂ are the same or different; and wherein C₁ and C₂ individually are chosen from a cysteine and a cysteine-like non-natural amino acid or amino acid analog.

The present disclosure also provides a tripeptide having the sequence XC₁C₂ and a metal; wherein the metal is complexed with the tripeptide; and wherein X is any natural or non-natural amino acid or amino acid analog such that XC₁C₂ and the bound metal are in a square planar orientation or square pyramidal orientation or both; and wherein C₁ and C₂ are the same or different; and wherein C₁ and C₂ individually are chosen from a cysteine and a cysteine-like non-natural amino acid or amino acid analog.

In addition, the present disclosure provides methods comprising complexing with a metal a tripeptide having the sequence XC₁C₂ to form a metal-XC₁C₂ complex; wherein X is any natural or non-natural amino acid or amino acid analog such that metal-XC₁C₂ complex has a square planar orientation or square pyramidal orientation or both; and wherein C₁ and C₂ are the same or different; and wherein C₁ and C₂ individually are chosen from a cysteine and a cysteine-like non-natural amino acid or amino acid analog.

Generally, the present disclosure is related to short, novel peptide motifs that strongly bind with a select metal, referred to as MAP tag(s). As such, these MAP tags can be used, among other things, to extract the select metal from a composition. The MAP tags of the present disclosure are 3 amino acids in length, and may be included in longer polypeptides and proteins at the N-terminus, C-terminus, or any position in between. In certain embodiments, however, it may be advantageous for the MAP tag to be present in a polypeptide or protein configuration that presents the MAP tag for binding with a metal, such as being present in an external loop. The MAP tag also may be attached to a non-peptide entity. Non-peptide entities include without limitation carbohydrates and/or covalent linkers. Additionally, more than one MAP tag may be present on a particular molecule. In one embodiment, the metal abstraction peptide (MAP) tag is a tripeptide capable of complexation with metal ions, as described in U.S. Patent Publication 2010/0221839.

Chemical Structure/Peptide Sequence

The MAP tags of the present disclosure generally comprise at least three contiguous amino acid residues capable of binding a metal. The MAP tags of the present disclosure generally have a sequence represented by XC₁C₂, in which C₁ and C₂ may be the same or different and may be a cysteine, or a cysteine-like non-natural amino acid, or a cysteine-like amino acid analog. For example, C₁ and/or C₂ may be a sulfur containing alpha- or beta-amino acid.

In certain embodiments, the MAP tag may be attached to another molecule. For example, the MAP tag may be attached to a non-peptide entity like a carbohydrate. For example, the carbohydrate may be a component in a glycoprotein. Alternatively, the carbohydrate may be hyaluronic acid or chondroitin. The attachment may be covalent, and may be affected through a linker.

In some embodiments, the MAP tag may comprise a sequence as follows: NC₁C₂; Z—NC₁C₂—Z¹; Z—NC₁C₂; NC₁C₂—Z¹; QC₁C₂; Z—QC₁C₂—Z¹; Z—QC₁C₂; QC₁C₂—Z¹; HC₁C₂; Z—HC₁C₂—Z¹; Z—HC₁C₂; HC₁C₂—Z¹; KC₁C₂; Z—KC₁C₂—Z¹; Z—KC₁C₂; KC₁C₂—Z¹; RC₁C₂; Z—RC₁C₂—Z¹; Z—RC₁C₂; or RC₁C₂—Z¹. Z may be any amino acid or any sequence of amino acids, and Z¹ may be any amino acid or sequence of amino acids that is equivalent or not equivalent to Z. Non-natural and amino acids analogues may be included as Z and Z¹.

In certain embodiments, a MAP tag of the present disclosure may be encoded in line with a gene or nucleotide sequence that provides for targeted delivery of the MAP tag, either before MAP tag complexation with a metal or after complexation with a metal. This may be accomplished using genes, peptides, or other motifs known to be useful for targeting. For example, MAP tags may be incorporated with an antibody, growth factors, peptides, and the like. Additionally, it can be incorporated into a peptide or protein using any synthetic or biosynthetic method for peptide or protein production. In some uses, the MAP tag spontaneously reacts with a metal to form a peptide-metal complex. Such peptide-metal complexes may form in solution or via transmetallation or any other process.

Metal Binding

Metal binding by MAP tags according to the invention can be accomplished using atoms in very close proximity, and as such, extreme conditions are required to release the metal. Thermal and chemical denaturation of the MAP tag and the material to which it is covalently linked permits slow release of the metal. For example, use of extreme conditions (e.g. boiling temperature, denaturants, chelators) may lead to slow release of the metal over a period of time (e.g., several to many hours).

In certain embodiments, the MAP tags of the present disclosure, alone or when incorporated into a polypeptide or protein, may complex with a metal to form a MAP tag-metal complex having a square planar/pyramidal geometry. The metal may complex with the MAP tag through 2N:2S coordination.

In general, the MAP tags of the present disclosure may bind, referring to IUPAC Group: Group 3 metals, such as Y; Group 5 metals, such as V; Group 6 metals, such as Cr, Mo, W; Group 7 metals such as Mn, Tc, Re; Group 8 metals, such as Fe and Ru; Group 9 metals, such as Co, Rh, Ir; Group 10 metals such as Ni, Pd, Pt; Group 11 metals, such as Cu, Ag, Au; Group 12 metals, such as Zn, Cd, Hg; Group 13 metals, such as Al, Ga, In, Tl; Group 14 metals, such as Sn and Pb; and Group 15 metals, such as Bi. In certain embodiments, the MAP tag binds to lanthanides or actinides, such as U. The MAP tag may bind and form a MAP tag-metal complex with Zn, Ni, Cu, Co, Pt, Pd, Au, Ag, Pb, and Fe.

In some embodiments, a MAP tag is capable of binding metals with high affinity. A MAP tag may also be capable of abstracting a metal from various compositions ranging from fluids to solids. Consequently, the ability of MAP tags to abstract the metal, rather than share coordination, make them amenable for use in separating a specific metal from another composition. In some embodiments, the MAP tags are capable of sequestering a metal ion from compositions by complexing with the metal and then abstracting or removing the metal from a component in the composition, such as a chelating agent (e.g., NTA) or a solid support conjugated with, for example, IDA or NTA. As such, the MAP tag is a metal abstraction peptide (MAP) tag. In various embodiments, binding is best accomplished using a partial chelator as opposed to a chelator that coordinates at all available binding sites on the metal. In various embodiments, chelators like EDTA that coordinate Ni and Cu at all available binding sites on the metal proceed much more slowly.

Site-Specific Modification

In various embodiments, the present invention relates to the use of specific natural and non-natural amino acid sequences for site specific modification of molecules of polypeptide origin. In some embodiments, the site specific modification is affected by a metal or metal ion. The metal or metal ion can be solubilized or partially or completely chelated. In some embodiments, the site specific modification comprises chiral inversion, e.g. chiral inversion of amino acids. In some embodiments, the site specific modification comprises cleavage. In various embodiments, the type of modification is controlled by selection of the metal and the conditions under which the metal-MAP tag complexation occurs.

Site-Specific Chiral Inversion of the Amino Acids

The present disclosure generally relates to MAP tags that demonstrate chiral inversion and/or have antioxidant activity, as well as methods of generating and using the same.

D amino acids that have been observed in proteins and peptides often impart significant differences in biological function to the chiral peptide and protein variants; while some may act as inhibitors, others may act as activators. The introduction of a D amino acid often alters the structure of the peptide or protein, which can in turn influence the biological activity of the parent peptide. Conversely, chiral mutagenesis of proteins has been shown to stabilize certain proteins, including insulin. Generally, naturally occurring, biosynthetic peptides and proteins that contain D amino acids are first generated entirely from L amino acids, and the inversion occurs as a post-translational modification.

In various embodiments of the invention, the metal-tripeptide (MAP tag) complex undergoes chiral inversion in a site-specific manner. This modification is distinctive, as site-specific chiral inversion is not often observed in proteins. The structural change that occurs in this complex can be both site and structurally specific and can occur substantially more rapidly than random background. Moreover, random chiral changes often require highly elevated temperature to occur and result in an approximately equal mixture of both chiral forms, rather than a single form. In various embodiments of the invention, the chiral inversion by the MAP-tag-based chemistry results in a substantially homogeneous single form.

In one embodiment, the present disclosure provides methods for site-specific chiral inversion of amino acids. Such methods may proceed at reactions occurring at room temperature and near neutral pH in aqueous solution over the course of hours. In general, the amino acids comprise at least three contiguous L-enantiomeric (L) amino acid residues capable of binding a metal. In various embodiments, MAP tags affecting or participating in site-specific chiral inversion generally have a sequence represented by XC₁C₂, in which C₁ and C₂ may be the same or different and may be a cysteine, or a cysteine-like non-natural amino acid (e.g., a sulfur containing alpha- or beta-amino acid), and in which C₂ also may be histidine, or a histidine-like nonnatural amino acid, and in which X may be another natural or non-natural amino acid or amino acid analog. In some embodiments, the peptide tag formed is capable of binding a metal in square planar/pyramidal geometry.

A particular molecule or complex of interest may comprise one or more of the amino acids represented by XC₁C₂ and may include additional amino acids (e.g., a peptide or protein), as well as non-peptide entities (e.g., carbohydrates).

In certain embodiments, the amino acids represented by XC₁C₂ may complex with a metal ion having a square planar/pyramidal geometry. The metal ion may complex with the MAP tag through 2N:2S coordination. In general, suitable metal ions include, but are not limited to, ions of nickel, zinc, cobalt, platinum, and palladium.

Prior to complexation with a metal ion, the amino acids represented by XC₁C₂ are L-enantiomeric. Upon complexation with a metal ion, one or more of the amino acids undergo a site-specific chiral inversion. For example, LXLC₁LC₂ may become DXLC₁DC₂ upon complexation with a metal ion provided X is a chiral amino acid. In another example, when X is an achiral amino acid, such as glycine, only C₂ may undergo the chiral inversion.

In one example, a MAP tag comprising the sequence Asn-Cys-Cys (NCC) may be formed through a reaction with IMAC resin-chelated nickel, which allows this NCC peptide to react with and abstract the metal, resulting in a high affinity Ni-peptide complex at neutral pH. The nickel-bound peptide, Ni-NCC, may contain a diamagnetic Ni^{II} center bound in square planar geometry with 2N:2S coordination.

In various contexts, the structure of Ni-NCC has mixed amine/amide nitrogen coordination and cis deprotonated thiolates. This coordination is very similar to that of Ni in nickel superoxide dismutase (Ni-SOD), which may consist of two cysteinate sulfurs (Cys2 and Cys6) arranged cis to one another, the N-terminal amine, and the deprotonated amide nitrogen from the peptide backbone of Cys2. When bound to nickel, the tripeptide can act a functional mimic of the enzyme nickel superoxide dismutase. Although a common function exists, the MAP amino acid sequence is not related to the sequence of the Ni-SOD enzyme participating in the same function.

In one example, the NCC tripeptide was synthesized to contain all L amino acids. Conversion to the D-containing form may occur after complexation with nickel resulting in inversion at two positions to produce a stable DLD-NCC-metal complex. In some examples, chiral inversion in NCC peptides occurs at the first and third Cα positions in the NCC sequence. The resulting DLD-peptide-metal complex is stable in aqueous solution and benefits from increase in thermodynamic stability. This inversion also imparts anti-oxidant chemistry to the complex.

In certain embodiments, the present disclosure provides anti-oxidant compositions and methods. For example, the metal ion-DXLC₁DC₂ complex has anti-oxidant properties. Such compositions may be targeted, or otherwise used, in applications or compositions benefiting from an anti-oxidant (e.g., therapeutic or cosmetic uses).

In another embodiment, the present disclosure provides methods and compositions for site-specific chiral inversion of amino acids that may be useful to protect against proteolysis.

In another embodiment, the present disclosure provides methods and compositions to modulate or enhance the solubility of a compound in water. For example, the metal ion-DXLC₁DC₂ complex is negatively charged. In general, the more a molecule is charged the more it is soluble in water.

In some embodiments, the metal abstraction peptide (MAP) tag is a tripeptide sequence capable of abstracting a metal ion from a chelator and binding it with extremely high affinity at neutral pH. Studies on the nickel-bound form of the complex demonstrate that the tripeptide asparagine-cysteine-cysteine (NCC) binds metal with 2N:2S, square planar geometry and behaves as both a structural and functional mimic of Ni superoxide dismutase (Ni-SOD). Electronic absorption, circular dichroism (CD), and magnetic CD (MCD) data collected for Ni-NCC are consistent with a diamagnetic Ni^{II} center. From the CD signal of Ni-NCC, the optical activity of the complex changes over time. Mass spectrometry data show that the mass of the complex is unchanged. Combined with the CD data, chiral rearrangement of the complex is seen to occur. Following incubation of the nickel-containing peptide in D₂O and back-exchange into H₂O, incorporation of deuterium into non-exchangeable positions is observed, indicating chiral inversion occurs at two of the alpha carbon atoms in the peptide. In total, these data indicate Ni-SOD activity is increased proportionally to the degree of structural change in the complex over time, as cross-correlation between the change in CD signal and change in SOD activity reveals a linear relationship. When MAP is embedded in a sequence such that the N-terminal amine is an amide, antioxidant chemistry is observed immediately with the metal ion-LXLC₁LC₂.

Site-Specific Peptide Cleavage

The present disclosure generally relates to methods for cleaving proteins in a site-specific manner and compositions for effecting such cleavage.

The present disclosure is based in part on the observation that protein cleavage may be affected through interaction of a metal (e.g., copper and iron) with the peptide sequence XC₁C₂/H (herein referred to as a metal activated cleavage (MAC) sequence). Accordingly, positioning this sequence in a protein (or its template nucleic acid sequence) allows site-specific protein cleavage.

Such site-specific cleavage may be useful in any number of applications benefiting from site-specific protein cleavage. For example, protein purification, sensors, analysis, diagnostics, therapeutics, prodrug, and enzyme activation (such as with a zymogen/proenzyme).

In some embodiments, the MAC sequences of the present disclosure are characterized by their ability to transiently interact with certain redox metals. In certain embodiments, the present disclosure provides methods comprising providing a peptide having a MAC sequence comprising XC₁C₂/H, wherein X is any amino acid (e.g., a natural or nonnatural amino acid or amino acid analogue), C₁ and C₂ are the same or different, C₁ and C₂ individually are chosen from a cysteine and a cysteine-like nonnatural amino acid (e.g., a sulfur containing alpha- or beta-amino acid); providing a partial chelator comprising a metal capable of reacting with the sequence XC₁C₂/H; and allowing cleavage of the peptide at the C-terminal portion of the sequence XC₁C₂/H.

The partial chelator may comprise a metal capable of reacting with the sequence XC₁C₂/H. Examples of suitable metals include, but are not limited to, redox metals, copper, and iron. The partial chelator is an assembly in which donor atoms surround a metal ion such that at least one coordination site remains available so that the metal may be accessed by the MAC sequence. The partial chelator should be capable of coordinating the metal, but not occupying all coordination sites around the metal. The coordination number (e.g., the number of bonds between ligands and the metal center) of a partial chelator may be 2, 3, 4, or 5-coordinate. Examples of suitable partial chelators include, but are not limited to, nitrilotriacetic acid (NTA), iminodiacetic acid (IDA), sulfate, histidine, and imidazole.

In certain embodiments, the cleavage rate of a MAC sequence may be tailored by controlling conditions such as pH, oxygen concentrations, anions, and by choice of the partial chelator. For example, higher pHs (e.g., 8-8.5) may increase the rate of cleavage. In some embodiments, the cleavage affected at pH 7.5, 7.6, 7.7, 7.8, 7.9, 8.0, 8.1, 8.2, 8.3, 8.4, 8.5, 8.6, 8.7, 8.8, 8.9, 9 or above. The cleavage rate also may depend on and be controlled by the particular amino acid chosen for the X position. Any of the natural and non-natural amino acids described herein and their suitable analogues are well known in the art and envisioned for selection in X position to modulate the cleavage rate.

The X in the MAC sequence may be any natural or non-natural amino acid. In some specific embodiments, X in the MAC sequence may be methionine (M), glucine (G), or asparagine (N). When an amino acid sequence is represented as a series of three-letter or one-letter amino acid abbreviations, it will be understood that the left-hand direction is the amino terminal direction and the right-hand direction is the carboxy terminal direction, in accordance with standard usage and convention.

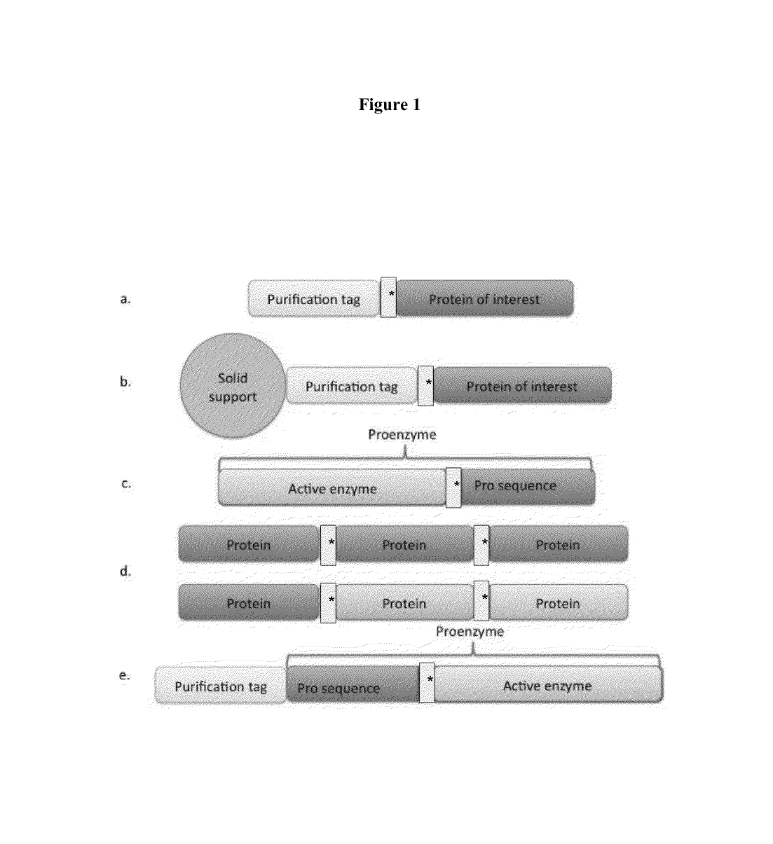

The MAC sequences of the present disclosure are 3 amino acids in length and may be included in longer polypeptides and proteins at the N-terminus, C-terminus, or any position in between (see FIG. 1in which * refers to a MAC sequence). In certain embodiments, however, it may be advantageous for a MAC sequence to be present in a polypeptide or protein configuration that presents the MAC sequence for binding with a metal, such as being present in a linker region between two proteins or in an internal sequence within a protein. A MAC sequence also may be attached to a non-peptide entity (e.g., polymer, fluorophore, solid support, chemical linker, and the like). Additionally, more than one MAC sequence may be present on a particular molecule.

While according to some embodiments of the invention, a MAC sequence of the present disclosure allows site-specific cleavage of the peptide at the C-terminal peptide bond, a MAC sequence also may be cleaved at the N-terminal peptide bond or within the MAC sequence itself.

A MAC sequence of the present disclosure can be encoded in line with a gene or nucleotide sequence for expression using any recombinant technology system. Additionally, it can be incorporated into a peptide or protein using any synthetic or biosynthetic method for peptide or protein production.

In certain embodiments, the present disclosure provides compositions that comprise a peptide having a MAC sequence comprising XC₁C₂/H, wherein X is any amino acid (e.g., a natural or nonnatural amino acid or amino acid analogue), C₁ and C₂ are the same or different, C₁ and C₂ individually are chosen from a cysteine and a cysteine-like nonnatural amino acid (e.g., a sulfur containing alpha- or beta-amino acid). In some embodiments, such compositions also may comprise an affinity tag in addition to MAC sequence. The affinity tag may be any affinity tag capable of binding to a substrate. The affinity tag may bind the substrate reversibly or substantially irreversibly. Examples of suitable affinity tags include, but are not limited to, maltose binding protein (MBP), glutathione-S-transferase (GST), poly(His), biotin ligase tags, Strep, HaloTag, cellulose binding domain (CBD), glutathione transferases (GST-tag), and a glycan.

In certain embodiments, in which the polypeptide may comprise an affinity tag, the present disclosure also provides compositions and methods that provide a substrate for the affinity tag.

In certain embodiments, in which the polypeptide may comprise an affinity tag, the present disclosure provides methods and compositions for improved purity of protein during purification through on-substrate cleavage. In such embodiments, the affinity tag and any contaminants that may be bound or retained by the substrate for the affinity tag are separated from the cleavage product of interest.

Protein Purification

In some embodiments, a target polypeptide or a target polypeptide-containing sample is treated to completely or partially purify some of the polypeptides/proteins for further analysis, and/or to remove non-protein contaminants. Any useful purification technique, or combination of techniques, can be used. Analytical and preparative methods are both envisioned and are within the scope of various embodiments of the invention.

Various embodiments of the invention relate to compositions and methods for the tagging of target polypeptides with an entity that is linked to the target polypeptide with a MAC sequence. The tags can be useful in various applications, including in protein identification and purification. Some embodiments comprise nucleic acid sequences encoding the constructs containing one or more tags, MAC sequences, and/or one or more target polypeptides. Nucleic acid sequences encoding compositions comprising one or more tags, MAC sequences, and/or one or more target polypeptides with specific designs discussed herein are comprised in various embodiments of the invention. In some embodiments, the tag is used as an affinity tag in a protein purification method.

In some embodiments, the use of the affinity tag for protein purification is combined with another protein purification method. Methods of purifying proteins are known in the art and include, by way of example, various chromatographic techniques, including, ion exchange, gel filtration/size exclusion, reverse phase, hydrophobic interaction, adsorption, affinity, and covalent; batch binding; precipitation; centrifugation; electrophoresis, including non-denaturing, isoelectric focusing, and two-dimensional; filtration and ultrafiltration; dialysis; reverse micellar extraction; concentration; lyophilization; and crystallization. Chromatography application may involve the use of high performance liquid chromatography.

Affinity based methods generally comprise matrix-immobilization of a ligand that binds to a target, which is generally insoluble. The matrix can either be precoated or provided in an activated form for the attachment of specific affinity ligands. Affinity reagents/materials commonly used in protein purification include, by way of example, antibody specific ligands, such as Protein A, Protein G and Protein A/G; antibodies; biotin and biotin derivative specific ligands, such as avidin and streptavidin; Nickel-NTA and cobalt immobilized metal affinity chromatography (IMAC) resins; glutathione and glutathione transferase (Glutathione S-Transferase; GST); maltose binding protein; poly(His); biotin ligase tags; Strep; HaloTag; cellulose binding domain; glycan and the like. In some embodiments, the examples of affinity reagents listed herein are used as affinity tags and a suitable binding partner is used as an affinity reagent on a matrix. Magnetic affinity separation particles, including paramagnetic particles and activated affinity purification beads can carry one or more ligands to be used as an affinity reagent/material during protein purification. In some embodiments, the affinity tags bind to affinity reagents/materials irreversibly. In some embodiments, the affinity tags can be reversibly eluted from the affinity reagents/materials. In some embodiments, the affinity reagents/materials are discarded after a single use, two uses, three uses, four uses, or five uses.

In various embodiments of the invention a poplypeptide is a chimeric protein. The chimeric protein may contain a binding domain with a known target. The chimeric protein can comprise an affinity tag. Chimeric proteins comprising a binding domain as affinity tags have been used in various ways, especially in protein purification and identification. For example, the chimeric protein composed of a desired protein fused with the C-terminal of a glutathione S-transferase is used widely, wherein the fused protein can be purified by a glutathione-Sepharose column. By means of the association between the binding domain of the chimeric protein and the substrate of the binding domain which is immobilized on a solid matrix such as beads, resins, plates, etc, the desired products can be conveniently purified.

In various embodiments, an affinity tag is linked to a target polypeptide using a MAC sequence. Constructs with affinity tags can be immobilized on a substrate that is capable of binding to the affinity tag. The immobilized constructs can be cleaved at the MAC sequence, releasing a portion of the target polypeptide from the substrate. The cleavage can be affected by contacting the MAC sequence with a cleavage agent comprising a partial chelator associated with a metal capable of reacting with the MAC sequence. In some embodiments, the cleavage agent comprises Cu(II). In some embodiments, the cleavage is affected at a terminal portion of the MAC sequence. In some embodiments, the cleavage is affected in the middle of the MAC sequence. In certain embodiments, the cleavage is affected at a site resulting in released polypeptide devoid of any of the MAC and/or affinity sequence. Various embodiments of the invention relate to compositions comprising the constructs discussed above. Further embodiments of the invention relate to composition comprising nucleic acid sequences encoding the constructs discussed above.

Methods and compositions comprising functionalized substrates with affinity reagents are known in the art. U.S. Patent Publication 2004/0002081, incorporated herein by reference in its entirety, discloses the use of monolithic substrate for the isolation and purification of polynucleotides. The use of the same for the isolation and purification of polypeptides is envisioned in various embodiments of the invention. U.S. Patent Publication 2010/0213411, incorporated herein by reference in its entirety, further discloses manufacture and use of monoliths for chromatography. U.S. Pat. No. 6,319,401, incorporated herein by reference in its entirety, discloses conversion of substrates both for analytical and preparative purposes in a flow-through and a cross flow reactor, a separation and/or conversion device, an analytical device as well as filtration device for carrying out such processes. U.S. Pat. No. 6,664,305, incorporated herein by reference in its entirety, discloses manufacture and use of chromatography materials, including monoliths, with functional groups, e.g. chromatography ligands such as affinity ligands. Affinity ligands comprising specific ionic interactions, such as ion exchange interactions, and affinity ligands comprising biospecificity, immunoaffinity, enzyme-substrate affinity, receptor-ligand affinity or nucleotide affinity, such as hybridization are further described. U.S. Pat. No. 6,736,973, incorporated herein by reference in its entirety, discloses porous self-supporting structures, wherein the surfaces of the pores are modified with functional groups, hydrophobic moieties, reactive groups for covalently binding of ligands, enzymes, immunoglobulins, antigens, lectins, sugars, nucleic acids cell organelles, or dyes. Affinity ligands, e.g. proteinaceous ligands, and functional groups comprising ion-exchange groups are also disclosed.

Nucleic acids encoding polypeptides or polypeptide fusion proteins/chimeric proteins described herein may be used to construct recombinant expression vectors capable of expressing the polypeptides or polypeptide fusion proteins/chimeric proteins of the present invention. In some embodiments, nucleic acid constructs capable of expressing the protein constructs described herein comprise nucleotide sequences containing transcriptional and translational regulatory information and such sequences are operably linked to nucleotide coding sequences.

A large number of suitable vectors are known in the art. Selection of the appropriate vector may depend on 1) whether it is to be used for nucleic acid amplification or for nucleic acid expression, 2) the size of the nucleic acid to be inserted into the vector, and 3) the host cell to be transformed with the vector. A vector may contain various components specific to its function (e.g. amplification of nucleic acid or expression of nucleic acid) and the host cell for which it is compatible.

In some embodiments, host cells are capable of expressing one or more polypeptides or polypeptide fusion proteins/chimeric proteins described herein. The host cells of the present invention encompass cells in procaryotic, eucaryotic, and insect cells. In some embodiments, host cells are capable of modulating the expression of the inserted sequences, or modifying and processing the gene or protein product in the specific fashion desired. For example, expression from certain promoters can be elevated in the presence of certain inducers (e.g., zinc and cadmium ions for metallothionine promoters). In some embodiments, modifications (e.g., phosphorylation) and processing (e.g., cleavage) of protein products are important for the function of the protein. Host cells of the present invention may have characteristic and specific mechanisms for the post-translational processing and modification of a protein. Suitable cell lines or host systems to ensure the correct modification and processing of the expressed protein are well known in the art. In some embodiments, host cells secrete minimal amounts of proteolytic enzymes. In some embodiments, host systems of viral origin are utilized to perform the processes described for host cells herein.

Various expression vector/host systems may be utilized equally well by those skilled in the art for the recombinant expression of polypeptides or polypeptide fusion proteins/chimeric proteins described herein. Such systems include but are not limited to microorganisms such as bacteria transformed with recombinant bacteriophage DNA, plasmid DNA or cosmid DNA expression vectors containing the desired nucleic acid sequence encoding polypeptides or polypeptide fusion proteins/chimeric proteins described herein; yeast transformed with recombinant yeast expression vectors containing the desired nucleic acid sequence encoding polypeptides or polypeptide fusion proteins/chimeric proteins described herein; insect cell systems infected with recombinant virus expression vectors (e.g., baculovirus) containing the desired nucleic acid sequence encoding polypeptides or polypeptide fusion proteins/chimeric proteins described herein; plant cell systems infected with recombinant virus expression vectors (e.g., cauliflower mosaic virus CaMV; tobacco mosaic virus, TMV) or transformed with recombinant plasmid expression vectors (e.g., Ti plasmid) containing the desired nucleic acid sequence encoding polypeptides or polypeptide fusion proteins/chimeric proteins described herein; or animal cell systems infected with recombinant virus expression vectors (e.g. adenovirus, vaccinia virus) including cell lines engineered to contain multiple copies of the nucleic acid sequence encoding polypeptides or polypeptide fusion proteins/chimeric proteins described herein, either stably amplified (e.g., CHO/dhfr, CHO/glutamine synthetase) or unstably amplified in double-minute chromosomes (e.g., murine cell lines).

In the case of cell or viral based samples, organisms may be treated prior to purification to preserve and/or release a target polypeptide. In certain embodiments, the cells are fixed using a fixing agent. In some embodiments, the cells are lysed. The cellular material may be treated in a manner that does not disrupt a significant proportion of cells, but which removes proteins from the surface of the cellular material, and/or from the interstices between cells. For example, cellular material can be soaked in a liquid buffer, or, in the case of plant material, can be subjected to a vacuum, in order to remove proteins located in the intercellular spaces and/or in the plant cell wall. If the cellular material is a microorganism, proteins can be extracted from the microorganism culture medium. Compositions and methods for polypeptide secreting microorganisms and other cell cultures are known in the art and can be applied when suitable. Alternatively, the polypeptides may be packed in inclusion bodies. The inclusion bodies can further be separated from the cellular components in the medium. In some embodiments, the cells are not disrupted. A cellular or viral poplypeptide that is presented by a cell or virus can be used for the attachment and/or purification of intact cells or viral particles.

Polypeptides may also be synthesized in a cell-free system prior to extraction.

In some embodiments, one or more protease inhibitors are included in the purification/extraction buffer(s). Representative examples of protease inhibitors include: serine protease inhibitors (such as phenylmethylsulfonyl fluoride (PMSF), benzamidine HCl, ε-Amino-n-caproic acid benzamide, and aprotinin (Trasylol)); metalloprotease inhibitors, such as EGTA (ethylene glycol bis(β-aminoethyl ether) N,N,N′N′-tetraacetic acid), and the chelator 1,10-phenanthroline; aspartate (acidic) protease inhibitors, such as pepstatin and diazoacetylnorleucine methyl ester (DAN); cysteine protease inhibitors, such as sodium p-hydroxymercuribenzoate; competitive protease inhibitors, such as leupeptin and antipain; and covalent protease inhibitors, such as iodoacetate and N-ethylmaleimide.

Cellulose Binding Domain

Various embodiments of the invention utilize a cellulose binding domain fused to a target polypeptide as an affinity tag. Cellulose can be used to bind to cellulose binding domains that are linked to target polypeptides for various applications including protein purification.

Cellulose, a major component of the cellular walls of plants, is a continuous linear glucose β-1,4 linkage polysaccharide which is readily available in the nature. Cellulase is a hydrolase of cellulose, wherein the cellulase can digest the cellulose by cleaving the β-1,4 glycosidic bonds of cellulose. The sequence encoding the cellulase comprises a cellulose-binding domain (CBD) allowing cellulose to bind to cellulose and subsequently cleave the β-1,4 glycosidic bonds of cellulose. U.S. Pat. No. 5,496,934, incorporated herein by reference in its entirety, discloses that cellulose binding domains have a high affinity for crystalline cellulose having a K_{d} ranging from 1.5 to about 0.8 μM. Affinity ranges from about 1.4 to about 0.8 μM, and from about 1.0 to about 0.8 μM are disclosed for pairings of CBD and crystalline cellulose. Chimeric proteins comprising CBD and a second protein retaining the avid binding capacity of the CBD to cellulose are also disclosed in U.S. Pat. No. 5,496,934, as well as nucleic acids encoding a cellulose binding domain. By means of the binding affinity between the CBD and the cellulose, the cellulase or CBD can be immobilized on the matrix coated with cellulose. Thus, with prices that are 100-500 fold lower than those of glutathione-Sepharose, cellulose provides an attractive, inexpensive matrix. Cellulose-based matrices further constitute safe materials that can be easily used in food and pharmaceutical industries.

Chimeric proteins with a cellulose binding protein have been disclosed in several U.S. patents and literature. For example, U.S. Pat. No. 5,202,247, incorporated herein by reference in its entirety, discloses a cellulose binding fusion protein having a substrate binding region of cellulase of Cellulomonas filmi origin. Cellulose substrates comprising carboxymethyl cellulose, microcrystalline cellulose, paper or cotton are also disclosed. Cellulose binding domains devoid of any linked cellulose activity are also described. U.S. Pat. No. 5,137,819, incorporated herein by reference in its entirety, discloses cellulose binding fusion proteins for immobilization and purification of polypeptides. U.S. Pat. No. 5,340,731, incorporated herein by reference in its entirety, describes a method of preparing a β-1,4-glycan matrix containing a bound fusion protein. U.S. Pat. No. 5,837,814, incorporated herein by reference in its entirety, discloses CBDs having a high affinity for crystalline cellulose and chitin, along with methods for the molecular cloning and recombinant production thereof. Fusion products comprising the CBD and a second protein are likewise described. Various applications for both the CBD and the fusion products, including drug delivery, affinity separations, and diagnostic techniques are contemplated.

Moreover, Wierzba et al. (Biotechnol. Bioeng. 47:147-154, 1995) discloses a chimeric protein which consists of the C-terminal binding domain of cellulase from bacteria and an amino acid sequence with the ability of cell-attachment. The chimeric protein can enhance cells anchorage on the matrix coated with cellulose. Proteins of the integrin family are disclosed for cell attachment, however any other suitable protein known in the art for cell attachment can be used.

In various embodiments, CBDs described herein refer to a protein comprising an amino acid sequence shown in U.S. Pat. No. 5,496,934, incorporated herein by reference, and includes functional homologs and functional derivatives thereof, provided that the functional homolog or functional derivative possesses the capability of binding to a cellulose-based material, e.g. cellulose-based matrix. In some embodiments, CBDs described herein bind to cellulose-based materials with high affinity and/or in a reversible manner. In some embodiments, the CBDs of the present invention are provided substantially free of other proteins with which it is naturally associated, for example, the balance of the major CpbA protein, discussed in U.S. Pat. No. 5,496,934. In some embodiments, one or more predetermined amino acid residues in the polypeptide may be substituted, inserted, or deleted, for example, to produce a CBD having improved biological properties, or to vary expression levels. Some of the desired CBD proteins falling within the scope of the present invention may optionally possess covalent or non-covalent modifications of the naturally occurring molecule, including, but not limited to, glycosylation modifications. In some embodiments, variant CBDs of the present invention, including those having residue deletions, substitutions and/or insertions may be prepared through the use of recombinant DNA technology, by altering the underlying nucleic acid. Various embodiments of the invention relate to compositions comprising nucleic acids encoding for variant CBDs. Variant CBDs in the chimeric constructs described herein, nucleic acids encoding thereof, and methods utilizing both are envisioned in various embodiments.

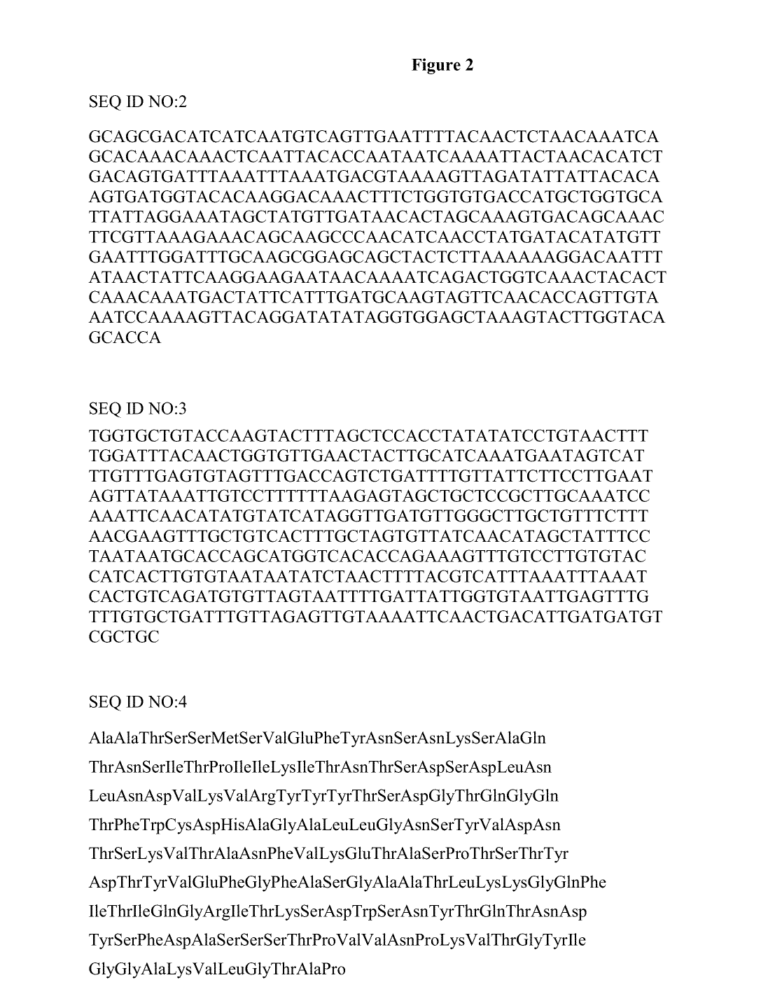

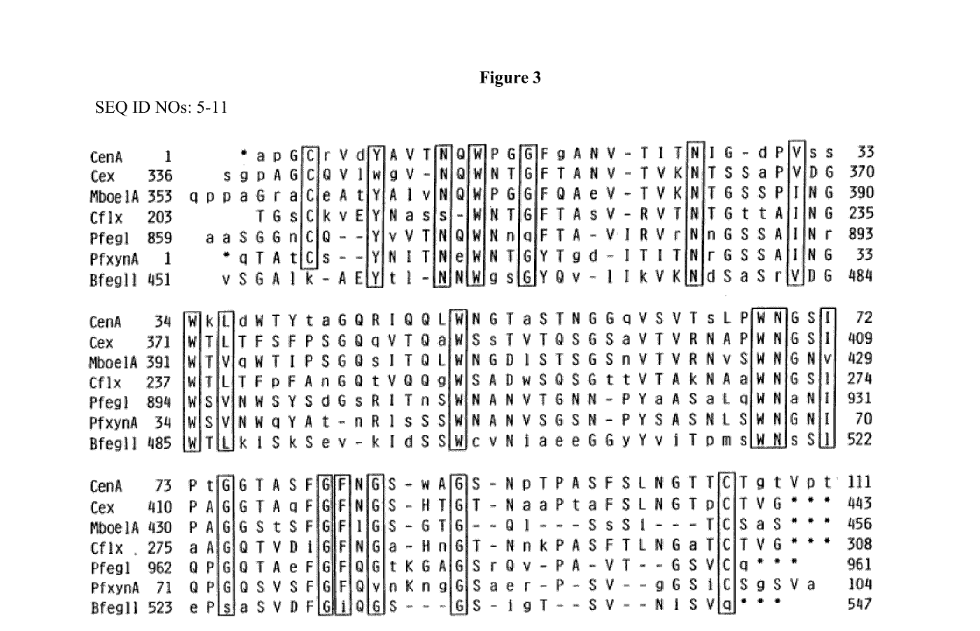

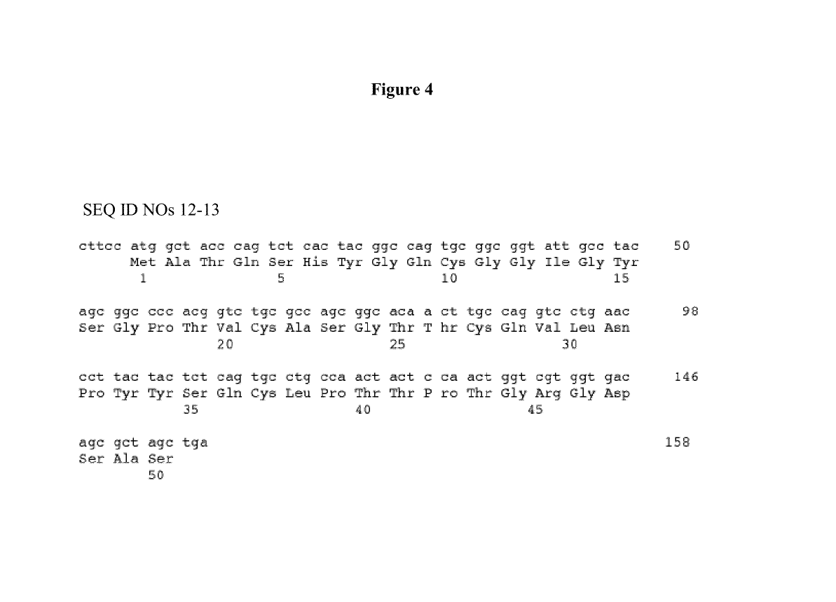

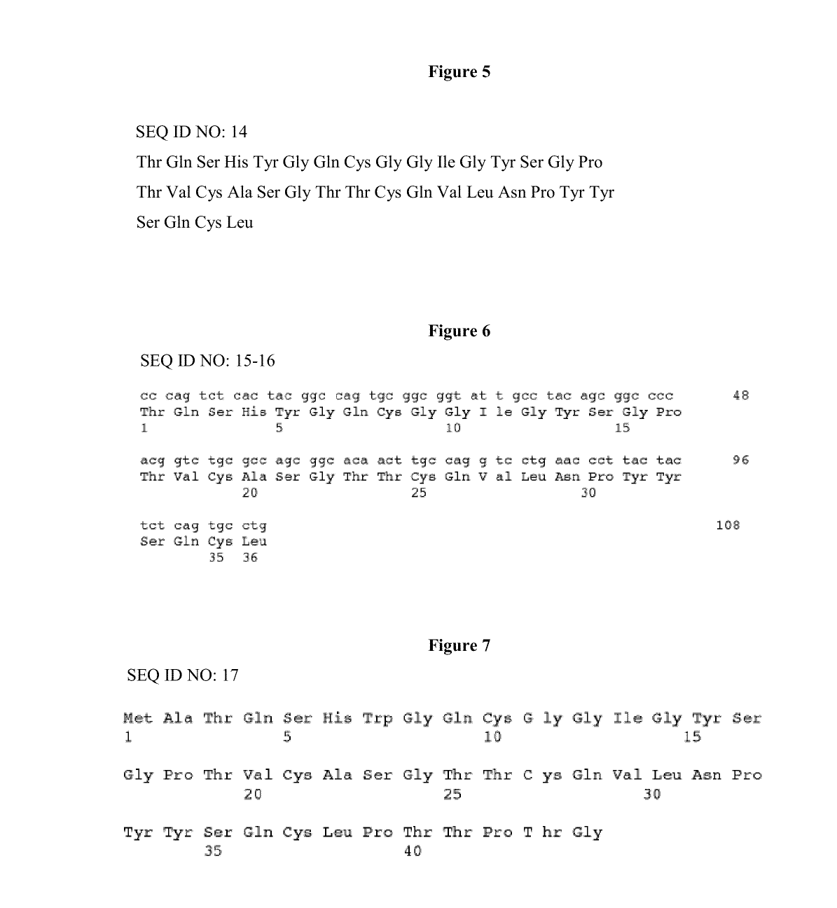

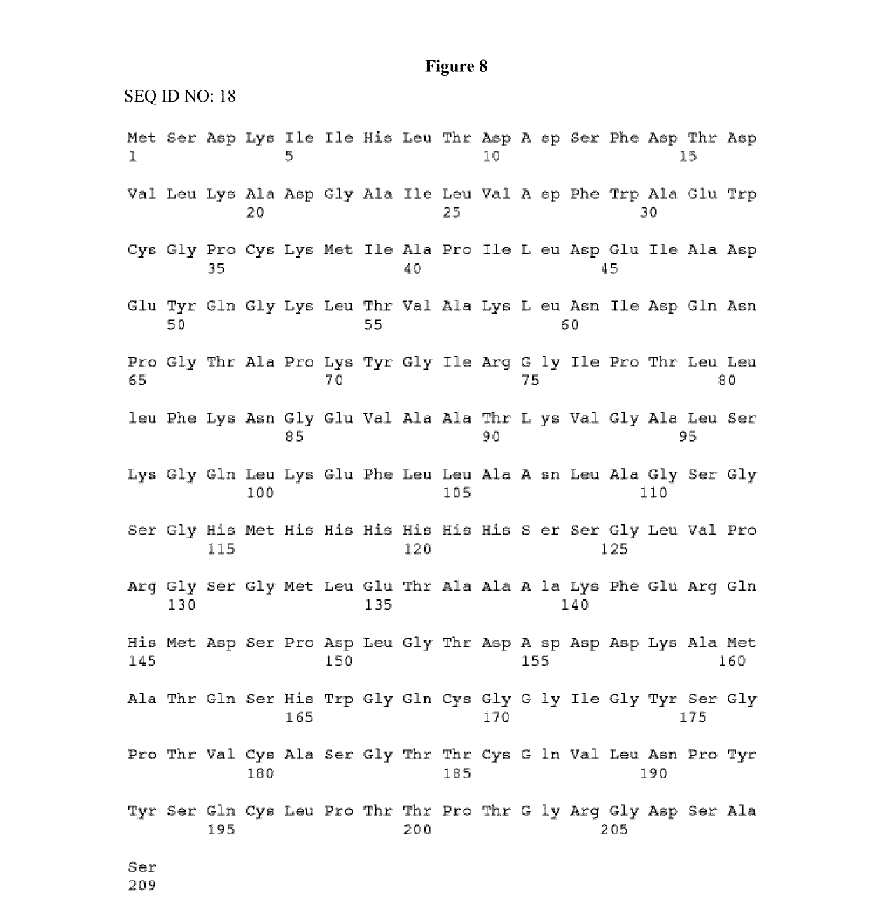

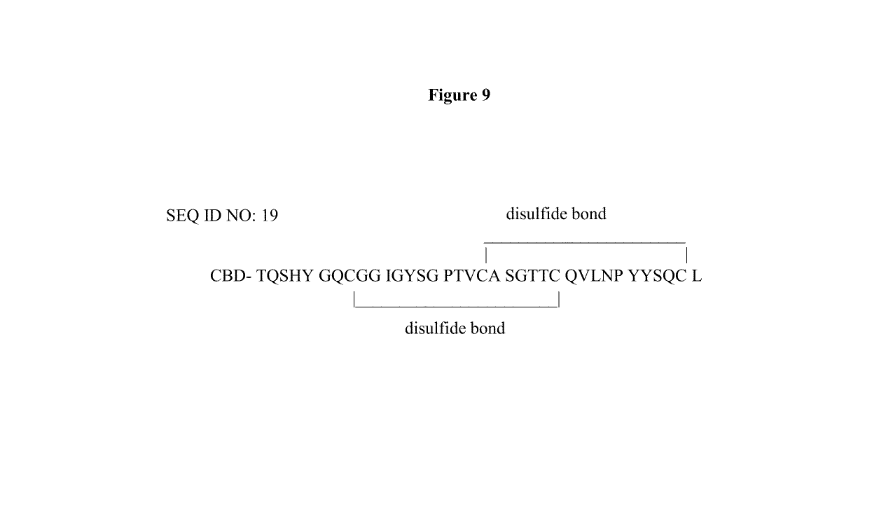

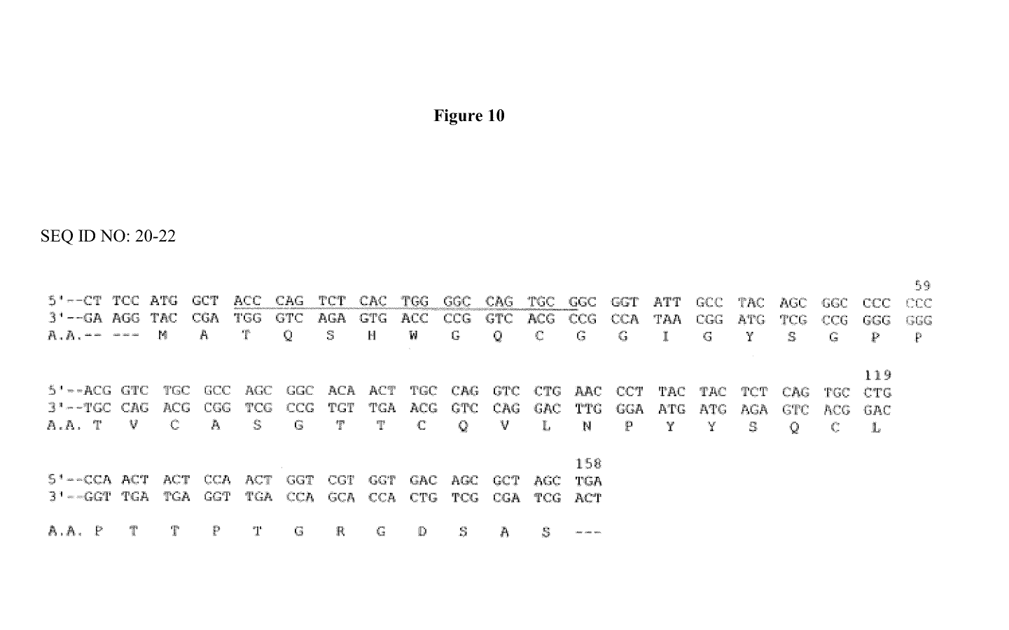

In some embodiments, the CBD is one having at least 70% sequence similarity, preferably, at least 80% sequence similarity, more preferably, at least 90% sequence similarity, and most preferably, at least 95% sequence similarity to one of the amino acid sequences shown in FIGS. 2-10 (SEQ ID NOs 4, 5-11, 13, 14, 16, 17, 18, 19, 22). The X % sequence similarity is also intended to include X % sequence similarity occurring in identified functional areas within the CBD protein of FIGS. 2-10. An example of a functional area would be a defined set of amino acids having the ability to bind cellulose with high affinity and in a reversible manner. Such protein homologs may also be referred to as CBD functional homologs. In one embodiment of the present invention, such a functional area may have about 100 amino acids. In another embodiment of the present invention, such a functional area may have about 50 amino acids. Various embodiments of the invention comprise nucleic acid sequences having at least 70% sequence similarity, preferably, at least 80% sequence similarity, more preferably, at least 90% sequence similarity, and most preferably, at least 95% sequence similarity to one of the nucleic acid sequences shown in FIGS. 2, 4, 6, and 10 (SEQ ID NOs: 2, 3, 12, 15, 20, and 21).

Various cellulose binding domains are primarily obtained from bacteria, wherein the cellulose binding domain located at the N-terminal of cellulase and the C-terminal catalytic domain are isolated from a proline- and threonine-rich amino acid sequence. Cellulose binding domains from other resources can be used as cellulose binding targets in various embodiments. U.S. Pat. No. 6,407,208, incorporated herein by reference in its entirety, discloses a cellulose binding domain from eumycetes. The eumycetes cellulose binding domain is shorter than many of the known cellulose binding domains from bacteria with a denser structure.

Thus, various embodiments of the invention relate to compositions comprising a polypeptide sequence sufficiently similar to or derived from such CBDs. Further, compositions comprising nucleic acid sequences encoding polypeptide sequences sufficiently similar to or derived from CBD sequences are also used in various embodiments of the invention. Methods for binding to chimeric proteins comprising a polypeptide sequence sufficiently similar to or derived from CBD sequences are provided herein. Further, some embodiments of the invention relate to compositions comprising a nucleic acid sequence encoding a CBD, wherein a nucleic acid sequence encoding a desired polypeptide can be or is linked/incorporated, such as an empty vector or a vector with an insert encoding a target polypeptide. In various embodiments, the linked nucleic acid product encodes for a CBD and a target polypeptide linked by a MAC sequence. Some embodiments of the invention relate to compositions comprising a chimeric construct comprising a CBD that is separated from the rest of the chimeric construct with a MAC sequence. In some embodiments, the construct is designed such that, upon a cleavage reaction, the entirety of the CBD is removed from the chimeric construct. In some embodiments, the construct is designed such that, upon a cleavage reaction, the entirety of the CBD and the MAC sequence is removed from the chimeric construct. In some embodiments, CBD is replaced with an alternative affinity tag in the constructs described above, such as a His-tag, a maltose binding domain, any other affinity tag described in this application or any other suitable affinity domain known in the art. Further embodiments of the invention comprise nucleic acid sequences encoding any of the specific chimeric constructs described herein.

### EXAMPLES

To facilitate a better understanding of the present invention, the following examples of specific embodiments are given. In no way should the following examples be read to limit, or to define, the entire scope of the invention.

### Example 1

Mutagenesis techniques were used to identify a peptide motif having a strong binding affinity with select metals. The human PRL-1 gene was cloned into pET-30 Xa LIC vector (Novagen) and mutated using the PCR-based QuikChange method (Stratagene). The primers generating the C170S, C171S, and H166A mutants, 5′ to 3′, were ggtcatagaaacaactCttgcattcaataaggatc (SEQ ID NO: 23, ggtcatagaaacaactgttCcattcaataaggctgtaactc (SEQ ID NO: 24), and cgtttcaaacattccaacggtGCtagaaacaactgttgcattc (SEQ ID NO: 25 (Integrated DNA Technologies, Coralville, Iowa), respectively. Capital letters indicate mutated bases. PCR reactions were treated with DpnI (Promega) for 1.5 h at 37° C., directly transformed into NovaBlue GigaSingles Competent cells (Novagen) and plated on LB with 30 mg/mL kanamycin selection. Individual colonies were grown overnight in selective LB at 37° C. The resultant DNA was purified using the Wizard Plus MiniPrep System (Promega). All mutations were confirmed by DNA dye-terminator sequencing (Northwoods DNA, Inc., Bemidji, Minn.). The double mutant C170S/C171S was produced by a second round of mutagenesis and similarly confirmed.

Vectors containing PRL-1 and relevant mutant genes were transformed into BL21(DE3) cells (Novagen) and grown overnight at 37° C. on selective LB. Selected colonies were grown in selective M9ZB broth overnight at 37° C. Overnight cultures were then transferred to 500 mL unlabeled minimal medium and induced with 1 mM IPTG at an OD₅₅₀ between 0.6 and 0.8. Proteins expressed for NMR experiments were grown in minimal medium containing ¹⁵N-ammonium chloride. Over-expression of the recombinant protein was confirmed by SDS-PAGE. Cells were pelleted by centrifugation at 4400×g and stored at −80° C. until used.

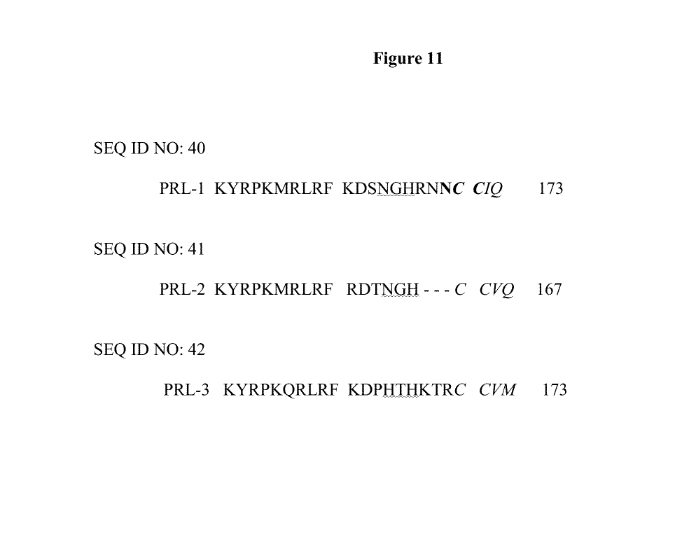

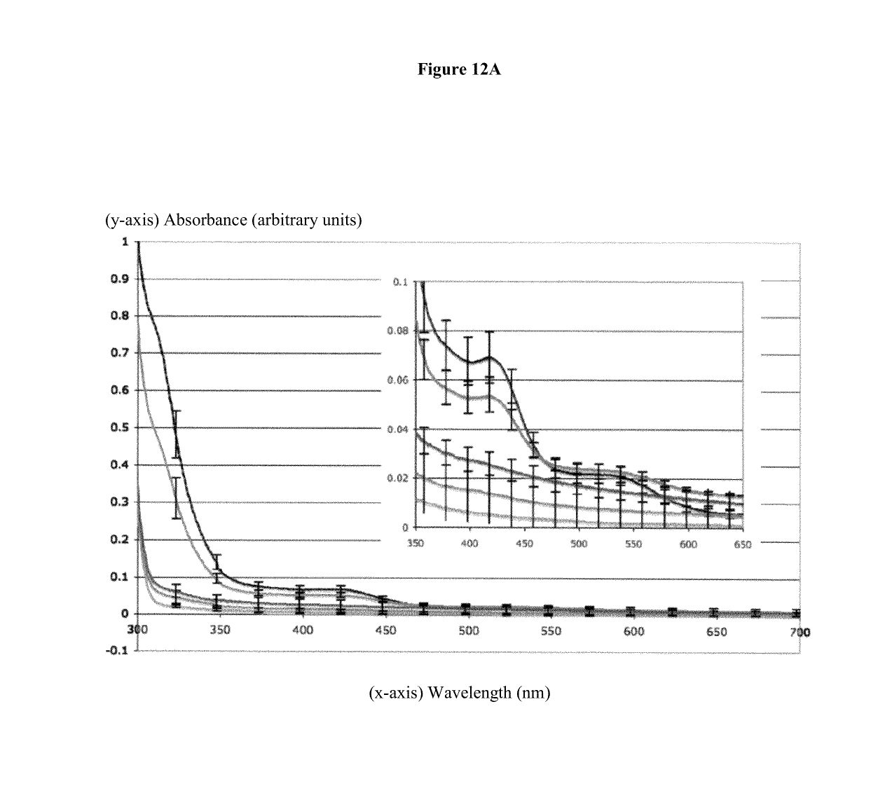

PRL-1 encodes NGH, a GGH-like consensus motif, near its C-terminus (FIG. 11. A series of PRL-1 mutants were examined to identify residues that participate in metal coordination. PRL-1 and PRL-1-H166A analyzed by ICP-MS in parallel bound approximately 120 and 60 μM Ni, respectively. Only a 20% decrease in signal was observed for H166A when measured by absorption spectroscopy. It also appears from the absorption spectra that the protein signal is diminished to an equivalent extent and that the loss of signal in the visible region is due to protein instability. Protein instability likely accounts for the disparity in the two detection methods since inductively coupled plasma mass spectrometry (ICP-MS) requires substantially more manipulation of the sample prior to analysis. Because the histidine mutant retained tight binding, mutations at C170 and/or C171 were made. Ni levels in individual cysteine mutants, as well as the double mutant, were greatly reduced relative to wild-type protein, indicating both Cys residues influence metal coordination. No Ni was detected in the C170S mutant or the C170S-C171S double mutant, but a small signal of approximately 17 μM was observed for PRL-1-C171S.

### Example 2

Protein purification techniques were used to purify the protein-metal complex. Cell pellets were resuspended in 30 mL buffer A (100 mM NaCl, 50 mM Tris®-HCl, 10 mM imidazole, pH 7.4, Ar-sparged) and lysed at 15,000 psi using a French pressure cell (ThermoElectron). Argon sparging of buffers was performed to displace oxygen. Samples then were centrifuged at 21,000×g and the supernatants were filtered through a 0.2 μm nylon filter. Purification of (His)₆-tagged PRL-1 was carried out using an Akta Explorer purification system at a flow-rate of 1 mL/min. Samples were applied to a metal-charged 5 ml HiTrap Chelating Column (GE Healthcare) and rinsed with 5 column volumes after loading. Elution was accomplished using a linear gradient to 60% buffer B (100 mM NaCl, 50 mM Tris®-HCl, 500 mM imidazole, pH 7.4, with or without argon sparging) over 13 column volumes, to 100% B over 3 column volumes, and maintained at 100% B for 4 column volumes. Elution was monitored by absorption at 280 nm, and fractions were examined for purity using SDS-PAGE. The appropriate fractions were pooled and dialyzed against Ar-sparged or not sparged 100 mM NaCl, 50 mM Tris®-HCl, pH 7.4. The (His)₆-tag was cleaved from the target protein using Factor Xa protease (Promega) overnight at room temperature. Protease was removed using Xarrest™ agarose (Novagen) and filtered with a 0.2 μm nylon filter before reapplication to the column to remove the tag and any uncleaved protein. The protein was applied to the column in dialysis buffer and the initial flow-through was collected to make the protein sample. Samples were concentrated and exchanged at least 10⁶-fold in Ar-sparged 100 mM NaCl, 50 mM Tris®-C1, pH 7.4 using (Amicon Ultra) 10 kDa MWCO centrifugal filters. Purity and mutant verification of the samples were ascertained by SDS-PAGE and ESI-MS, respectively. Columns were treated with 1 M NaOH, stripped with 2 column volumes of 100 mM EDTA and recharged with Ar-sparged 100 mM NiSO₄, 100 mM CuSO₄, 100 mM CuCl₂ or 100 mM ZnCl₂ immediately preceding each use.

Metal bound to the His-tag has octahedral geometry, which is blue-green in color and the absorption corresponding to such a coordination event would appear in the 700-800 nm region of the spectrum. However, WT PRL-1 purified in the presence of Ni(II), pH 7.4 shows a characteristic rust color.

The rust color of purified wild-type PRL-1 protein could result from metal complexation or oxidation of aromatic residues (i.e., Trp or Tyr). Oxidation of aromatic residues was ruled out based on the mass spectrometry data from the intact and tryptic digest studies of the protein. In addition, the density of the protein was higher than predicted, as the rust-colored protein becomes concentrated at the bottom of the tube during high-speed centrifugation, where metal coordination would increase the density of the protein complex.

When cell lysate containing recombinant WT PRL-1 was equally divided and purified side-by-side using Ni²⁺-, Cu²⁺- or Zn²⁺-charged IMAC resin, the protein abstracted the metal bound to the chelating resin. The rate of transmetallation was most rapid with nickel, followed by copper. Zinc was present to some extent in all samples, indicating the protein picked up zinc in vivo. When zinc was bound to the column, only zinc was observed in the protein. Apo PRL-1 was poorly soluble and loss of metal leads to precipitation. The soluble material from each purification when tested by ICP-MS had an equivalent proportion of metal bound.

### Example 3

ICP-MS was used to characterize the bound metal. Purified protein samples were digested in concentrated nitric acid in PFA microcentrifuge tubes (Savillex, #s 7240, 7241) at 68±1° C. for 16.5±0.7 hours. Based on protein quantification prior to digestion, the sample was then diluted to 1.5 μM in 20 mM ammonium bicarbonate, pH 8.2, filtered through a 0.2 μm nylon filter, and injected into a VG Elemental VGII+XS Inductively-Coupled Plasma-Mass Spectrometer fitted with a microconcentric nebulizer. A minimum of two separately prepared samples were analyzed for each protein variant. Each sample was scanned twice and standards were run before, between, and after each sample scan. Drift was monitored during washes between scans of both sample and standard injections.

To confirm that the chromophore arises from protein abstraction of the metal from the charged IMAC matrix, samples were subjected to ICP-MS for semi-quantitative analysis. As extensive washing is used to remove background metals, the values obtained from this method inherently underestimate the amount of metal bound, but can be used to assess the relative affinity of different metals for the protein. Additionally, to identify an endogenous metal ligand that may have been bound to PRL-1 during expression in the cells, ICP-MS was used to look for the presence of a transition metal from the first two rows in the periodic table as well as other common biologically relevant metals. The data showed that PRL-1 bound only to Zn and the specific metal, for example Ni, used on the IMAC column during purification. On average 500 μM Ni-purified protein samples were digested in nitric acid for 16 h at 70° C. were determined to contain approximately 180 μM Ni, yielding a Ni to protein stoichiometry of 1:2.7+/−0.7. Zn was also detected in the Ni-purified samples at approximately 60 μM. Separately, purification of PRL-1 using Zn-IMAC showed Zn was the only metal present in high quantity at 270 μM [1 Zn:1.8 protein], and these protein samples contained only background levels of Ni. ICP-MS of both Ni- and Zn-purified PRL-1 showed no other metals in significant quantity, which suggests that zinc is coordinated in vivo and becomes partially displaced by Ni²⁺ during the nickel chromatographic purification. Because ICP-MS analysis is conducted in a flow of Ar and the most abundant isotope of Ar has the same mass as Ca, Ca binding cannot be determined from the primary mass signal. Nonetheless, because a much higher amount of Ca was present in the cell culture media compared to Zn and yet the ICP-MS signal corresponds largely to zinc, the protein is unlikely to bind calcium. The ICP-MS data indicate Zn was present in an appreciable amount when PRL-1 was purified using Ni-IMAC, which suggest that Zn binding occurred in the cell.

### Example 4

UV-Vis absorption spectra were used to measure the concentration of the PRL-1 protein. The concentrations of purified PRL-1 analogs were measured at 280 nm and calculated using an extinction coefficient of 19420 M⁻¹ cm⁻¹. Because of possible metal-protein absorption interferences in the high 200 nm region, the validity of the UV-Vis quantification method was confirmed by the Bradford method. Spectra of PRL-1 analogs at concentrations of 10, 5, 2.5, and 1.25 mg/mL were collected on a Cary 100 UV-Vis spectrophotometer from 800 to 200 nm. To correct for scattering effects in the analysis, PRL-1-WT spectra were adjusted by subtracting the PRL-1-C170S-C171S spectrum, because it does not bind metal. The resulting spectra were resolved and peak positions were evaluated using GRAMS/AI 7.00 software. Three methods were aimed at metal displacement. In the first, PRL-1-WT (e.g., wild type) samples were reduced using 20 mM β-mercaptoethanol (BME), and imidazole was added to a concentration of 10 mM. For 0.5 mM protein samples, this corresponds to 400-fold solution excess of BME to compete with the two C-terminal cysteine thiols and a 200-fold solution excess of imidazole to compete with the single C-terminal histidine imidazole side chain. New baselines were taken and the spectra recalculated. In the second method, baselines were recalculated for buffer containing 100 μM EDTA, and the spectrum for the PRL-1-WT sample that was exchanged ten million fold in 100 μM EDTA was recorded. Finally, 10 mg/mL protein samples were denatured in 5.6 M guanidinium hydrochloride and heated to 90° C., new baselines were taken, and the spectra were collected at 0, 2, 4 and 6 hrs.

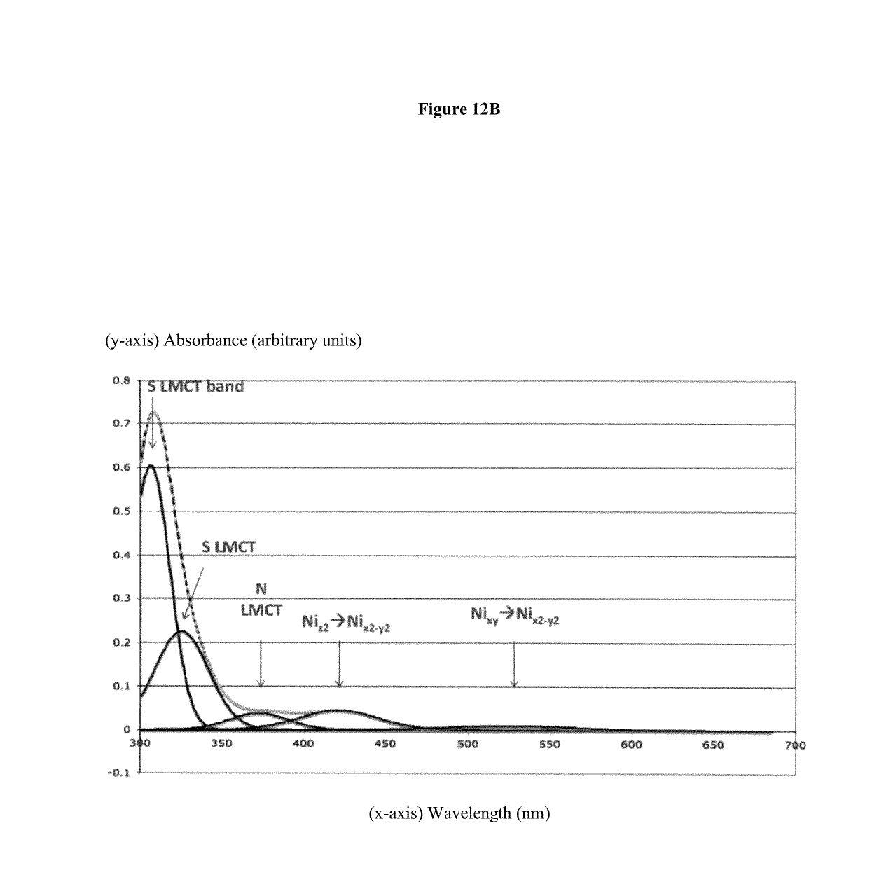

Accordingly, characterization of metal coordination by PRL-1 was determined with UV-Vis absorption spectroscopy. The metal coordination geometry employed by PRL-1 and its mutants was analyzed using UV-Vis absorption spectroscopy at 800-200 nm. Absorption spectra of PRL-1 produced peaks at 280 and 220 nm, which arise from aromatic and peptide bonds within the protein and correspond well with the concentration determined using the Bradford assay. Metal-containing compounds often absorb at several wavelengths in the visible range, and the Ni- and Cu-purified PRL-1 proteins also display peaks in the visible range, which correspond to the rust color of the sample. PRL-1 WT shows a maximum at 318 nm (evident as a shoulder on the very large 280 band), a broader signal at 421 nm, and a very broad peak at 526 nm (FIG. 12A). Cu-purified PRL-1 generated a similar spectral profile as Ni purified PR1-1; however, a small blue shift was observed for the individual spectral peaks, which was expected when Cu is substituted for Ni. No absorption or visible color was observed when the protein was purified using resin charged with Zn, where Zn(II) has a completed d-shell and as such should not show electronic transitions in the visible spectrum.

Relative to the WT protein, the H166A variant showed the same absorption profile, but the peaks were decreased in intensity to a degree consistent with the ICP-MS results. The spectra of PRL-1-WT and PRL-1-H166A were distinct from those of the cysteine mutants in the visible region. At equivalent concentration, individual substitutions at cysteine 170 and/or 171 with serine showed complete loss of signal at 421 and 526 nm. These mutant proteins were examined at concentrations as high as 10 mg/mL (0.5 mM), and no specific absorption bands were detected. Signal from the WT is evident at 0.1 mg/mL, indicating that binding of Ni was decreased by each mutation at least 100-fold.

Deconvolution of the UV-Vis spectra of corrected WT protein was performed between 300 and 700 nm and revealed that the 318 nm peak was composed of 3 separate absorbance bands at 306, 325, and 372 nm, respectively contributing 54%, 38% and 7% to the total composite peak absorbance (FIG. 12B). The absorption profile of PRL-1-H166A was highly similar to the wild-type protein spectrum. The absence of the imidazole group at residue 166 does not shift the absorbance maxima or contribute significantly to the component bands of the 318 nm absorption, further indicating that the imidazole from the histidine within the NGH motif does not directly coordinate Ni.

Ni binding in PRL-1 was not affected by reduction of the disulfide bond at PRL-1's active site. Addition of 20 mM BME did not alter the UV or visible region of the spectral absorption profile, indicating that metal binding is largely unaffected by reduction of the protein and the presence of the reducing agent. Subtraction of the reduced spectrum from the oxidized confirms that there was no significant difference in metal coordination between the reduced and oxidized WT samples. To examine whether a small contribution from a His is made, additional imidazole was added to the solution to increase the signal strength of a transient association. Addition of 10 mM imidazole had no effect on the absorption profile, which suggests that access to the Ni is limited. Additionally, no spectral change was observed when the Ni-purified sample was reduced in the presence of imidazole. Ten million-fold exchange of Ni-purified WT sample into buffer containing 100 μM EDTA had little effect on the spectrum, and absorbance values for this sample were within error of the WT absorbance. Raising the pH from 7.4 to 8.5 should increase the molar absorptivity coefficient (ε) by approximately 6-fold, but no change in ε was observed when the pH was elevated, which may suggest that the bound Ni is solvent inaccessible in PRL-1. Decreasing the pH to 6.5, however, decreased the intensity of the visible peaks.

Ni binding appeared to be irreversible at pH 7.4, as displacement of Ni from Ni-purified PRL-1 required aggressive measures. When Ni-purified PRL-1 was treated with 5.6 M guanidinium hydrochloride at 90° C., the peaks at 318, 421 and 526 nm only gradually disappeared over 6 hrs. The concomitant appearance of a signal at 750 nm was observed over the 6 hours, which indicates Ni was released from the protein and coordinated by water in octahedral geometry.

FIG. 12A shows the electronic absorption spectra of PRL-1 variants purified in the presence of Ni²⁺ ion. Three spectra for each protein variant were recorded from 800 to 200 nm and averaged. Protein concentration for all variants is 5 mg/mL to within 10% based on A₂₈₀ comparison. Shown are the spectra from 700 to 300 nm. Visible features are absorbance maxima at 318 and 421 nm.

FIG. 12B shows a resolved spectrum of PRL-1 WT. Resolution of the UV-Vis spectrum of Ni-purified PRL-1 WT protein between 300 and 700 nm fits the data using 5 curves. The 318 nm shoulder is resolved into peaks at 306, 325, and 372 nm. The sum of the resolved absorbance peaks (dashed line) and the raw data (solid line) are both shown. The peaks at 421 and 526 nm are also present. The inset of FIG. 12B shows the spectra from 600 to 400 nm are shown at an expanded scale for visualization of absorption maxima at 421 and 526 nm.

### Example 5

Electrospray ionization mass spectrometry (ESI-MS) was used to analyze the samples. Briefly, ESI spectra were acquired on a Q-T of-2 (Micromass Ltd, Manchester UK) Hybrid Mass Spectrometer operated in MS mode and acquiring data with the time of flight analyzer de-tuned to 8000 resolution (FWHH) for sensitivity. For whole protein ESI spectra, purified protein samples were diluted to 1 μg/μL in untreated (“oxidizing”) 20 mM ammonium bicarbonate, pH 8.2 and allowed to incubate at room temperature. Time points were taken on the first, fourth, and seventh day post-purification and frozen at −80° C. until ready for use. Samples were desalted on a short (3 cm×1 mm I.D.) reverse-phase (RP) HPLC column (Hamilton PRP1, Reno, Nev.). Samples were loaded onto the column from a 1% formic acid solution with protein (5 μg), washed in same solution, and eluted with 90% MeOH/0.5% formic acid directly into the ESI source. The cone voltage was 60 eV, and the voltage on the collision cell was 20 V. Spectra were acquired over the mass range 800 to 3000 u, accumulating data for 5 seconds per cycle.

For tryptic digests, PRL-1-WT, PRL-1-H166A and PRL-1-C170S-C171S were diluted to 1 mg/mL in ammonium bicarbonate, pH 8.2, with or without 20 mM β-mercaptoethanol, and trypsin (Promega) was added in a 1:50 ratio protease:protein (w/w) and incubated at 37° C. for 16 hrs. To confirm complete digestion, samples at various time points were run on SDS-PAGE and visualized by silver staining Coupled to MS analysis, capillary HPLC separations were performed using a Zorbax SBC18 RP column (5 cm×0.32 mm I.D., 3.5 μm bead size, 300 Å pore size) packed by Micro-Tech Scientific (Sunnyvale, Calif.), with a chromatograph (Waters capLC XL, Milford, Mass.) that develops gradients at 10 μL/min. A linear gradient of 20 to 80% B was applied over 120 minutes. The solvents were: A 99% H2O, 1% MeOH, B 99% MeOH, 1% H2O, both 0.08% formic acid. Argon was admitted to the collision cell at a pressure that attenuates the beam to about 20%. This corresponds to 16 psi on the supply regulator or 5.3×10⁻⁵ mBar on a penning gauge near the collision cell. The collision cell was operated at 8 V for maximum transmission, and spectra were acquired over the range 250 to 2000 u, accumulating data for 8 seconds per cycle.

NMR experiments were conducted to analyze the samples. Briefly, 2D ¹H-¹⁵N HSQC spectra were acquired on a Bruker Avance 800 MHz NMR spectrometer using a cryogenic, triple-resonance probe equipped with pulse field gradients. Water suppression was accomplished using flip-back pulses. All spectra were obtained at 37° C. and acquired in 16 scans with 2048 points in ¹H. 256 or 128 increments were collected in ¹⁵N for WT and C170S-C171S, respectively. Samples were prepared in 50 mM sodium phosphate, 100 mM NaCl at pH 6.5 and contained 5% D₂O. The concentration of WT PRL-1 and PRL-1-C170S-C171S was 1.0 mM and 0.8 mM, respectively. Sample reduction was accomplished by addition of 10 mM DTT at least 24 hours prior to spectral acquisition. ¹H chemical shifts were referenced with respect to an external DSS standard in D₂O. Indirect referencing relative to ¹H was determined for ¹³C and ¹⁵N, assuming ratios ¹³C/¹H=0.251449530 and ¹⁵N/¹H=0.101329118. Computer programs nmrPipe and Sparky were used for data processing and spectral analysis.

### Example 6

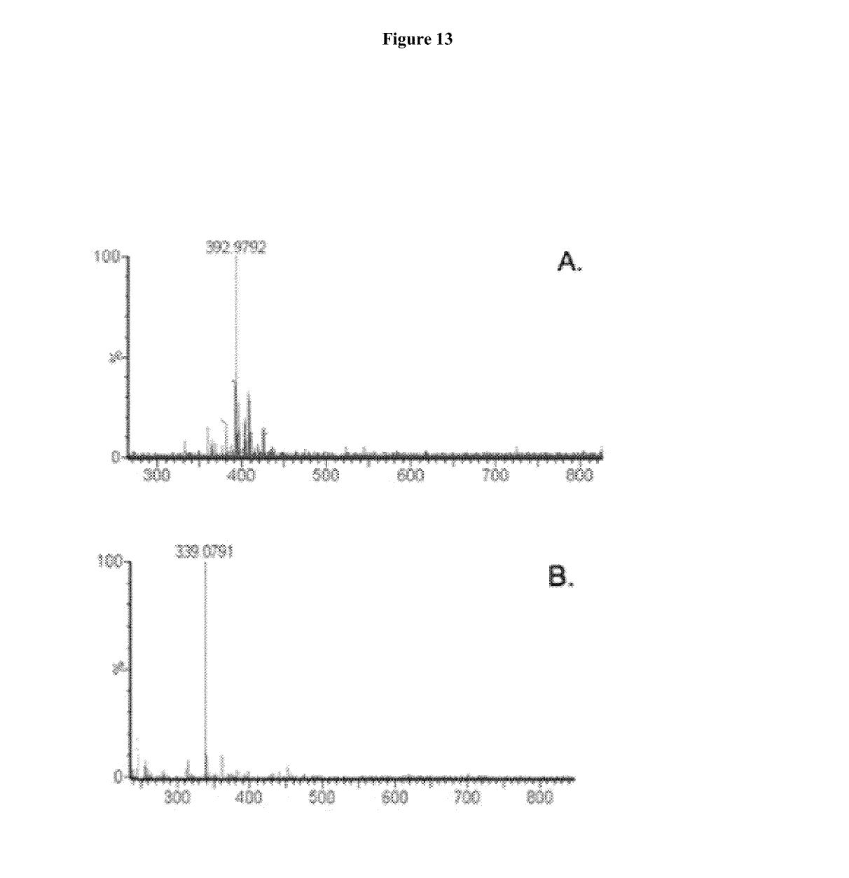

To confirm that the MAP tag motif was entirely responsible for metal binding, the NCC peptide was synthesized and purified, and the peptide was incubated with metal-charged IMAC resin. Complex formation was verified using electrospray ionization mass spectrometry (ESI-MS) operating in negative ion mode (FIG. 13).

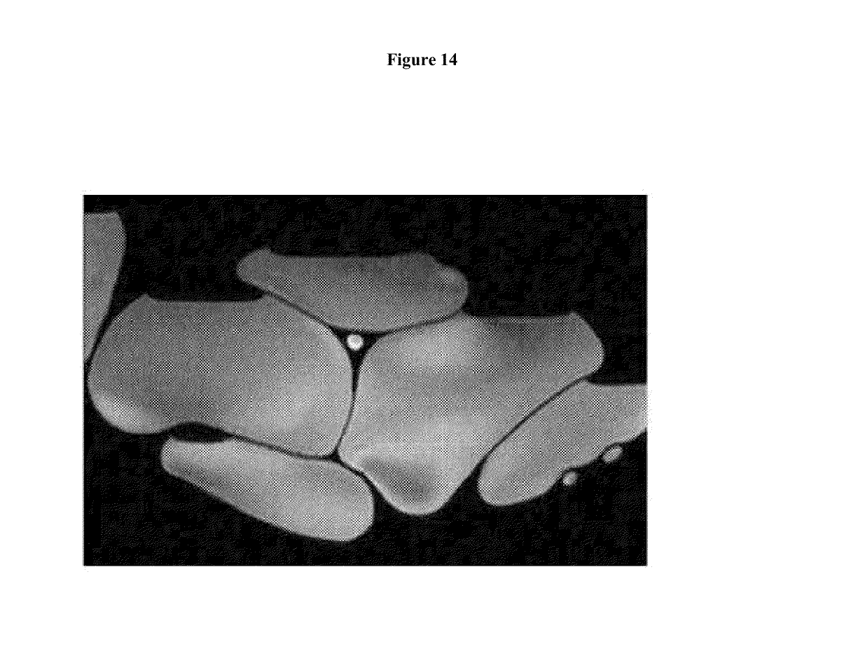

In the NCC peptide, the absorption signal mimicked that observed for the peptide-metal complex embedded in PRL-1, but the complex was more exposed in the tripeptide. The absorption in the visible range intensified when the pH was raised from 6 to 7.4. Acidification diminished the signal intensity and when returned to the more basic pH, full signal fails to be restored, indicating irreversible loss of some metal from the square planar geometry may occur. Elevation of the pH to 10 suggested a fifth ligand, presumably water or hydroxide, is coordinated, generating a square pyramidal arrangement. At pH 7.4, this ligand is presumably highly exchangeable and too transient to be detected by absorption spectroscopy. The exchangeable ligand provided an avenue for paramagnetic relaxation of the bulk solvent and can be detected by MRI (FIG. 14). Additionally, the metal was added by mixing the metal salt into aqueous solution containing the peptide. Depending on the conditions, the absorption spectrum of the peptide either paralleled the complex formed via metal transfer or was distinctly different from that obtained for the full-length protein. The differing spectrum reveals that the metal may be coordinated solely by sulfur in this case. Although the NCC peptide is sufficient to confer metal binding, the type of complex generated depends on the method by which the metal is introduced to the peptide. Transmetallation provided an efficient pathway for incorporation of the metal into the NCC tripeptide to generate the extremely high affinity square planar complex.

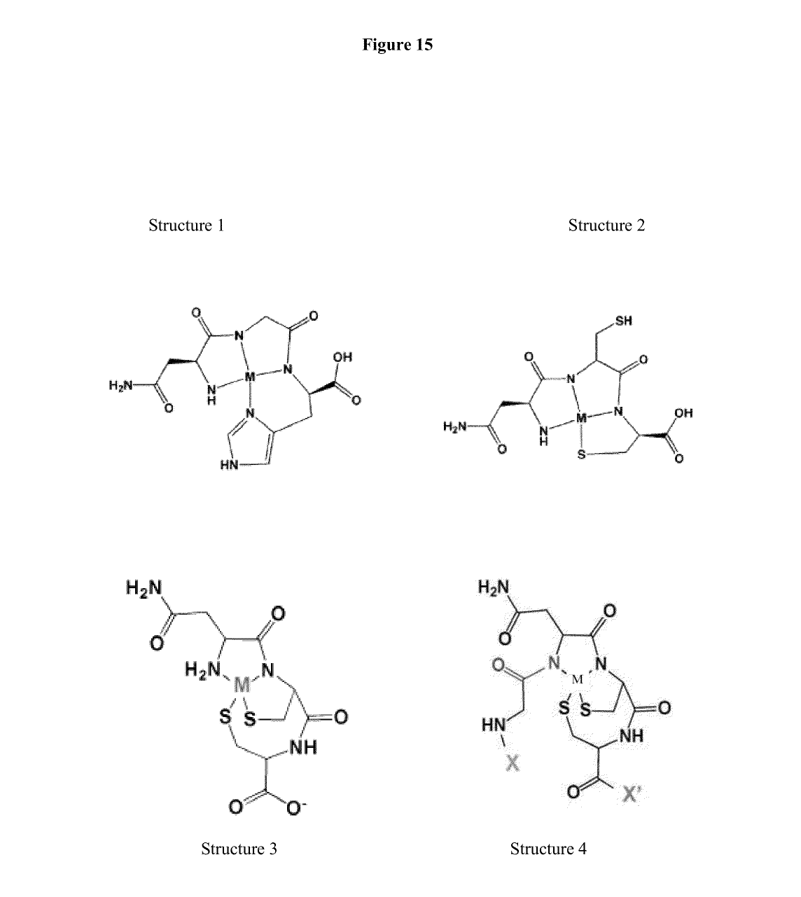

The GGH-like metal complex of NGH is shown in FIG. 15, Structure 1. A possible chemical structure for the metal-bound NCC complex is shown in FIG. 15, Structure 2, which corresponds to a GGH-like arrangement that involves coordination of the metal by several deprotonated backbone N atoms. In this case, the imidazolium nitrogen from His would be replaced in NCC with the sulfur from the Cys in the third position in the coordinated complex. This complex would have 3N:1S coordination, which is not consistent with absorption spectra. To verify the unique, specific coordination by NCC, NGC also was synthesized and confirmed the importance of the central Cys side chain in metal binding. The NGC peptide incubated with the IMAC resin did not produce an absorbance spectrum that resembles the metal-bound NCC or PRL-1 spectrum. Therefore, GGH-like coordination is not utilized and must have a much lower affinity for binding metal in the specified geometry.

Structure 3 does not correspond to a GGH-like structure and is consistent with the data obtained for both the peptide alone and the peptide in the context of the larger protein. The absorption spectrum of both indicate that two sulfur atoms participate in the complex along with two nitrogen atoms, one which is deprotonated and the other which has a single proton attached. Structure 3 may not involve the N-terminal amine group, but instead may utilize an amide group. Involvement of an amide (FIG. 15, Structure 4) would cause differences in the spectra of the peptide and protein because this atom would be embedded in a peptide bond in the protein, which is also consistent with absorption spectra.

UV-Vis spectra derived from Ni-purified WT PRL-1 display bands near 318, 421 and 526 nm. For peptides and organic molecules binding Ni in a square planar geometry via nitrogen ligands, characteristic d-d bands near 420 nm are reported in the literature. Separately, the reduced, square-planar form of NiSOD was analyzed using density functional theory, the results of which show similar relevant features. The locations of the bands as well as the trend in their relative intensities in these systems are well preserved in the PRL-1 protein spectrum. The strong 318 nm band reflects the ligand-metal charge transfer band (LMCT). Resolution of the spectrum reveals that this absorbance is likely a composite of 3 bands at 306, 325, and 372 nm. Using the other structures as a guide, the sharpest, most intense band at 306 nm would correspond to transitions from sulfur atoms, with the other two bands likely arising from nitrogen donation or possibly weaker sulfur donation.

Within the series of C-terminal mutants, only mutations involving C170 and C171 abolished signal at these wavelengths. Most notably, the 318 nm envelope corresponding to the LMCT is lacking in the cysteine mutant spectra. With respect to the entire absorption profile, the small very broad absorbance in the low 300 nm region for the cysteine mutants appears to be an artifact from high-intensity bands at lower wavelengths or a scattering effect, as it is positioned incorrectly to be one of the 318 nm component bands. The bands attributable to transitions within Ni d-orbitals (421 nm, 526 nm) also are absent when sulfur is substituted by oxygen. The original bands are absent and no additional bands appear upon mutation of either C170 or C171 in the absorption spectra. Also no disulfide bonds involving these residues are observed in the MS data. As such, it seems that both cysteines directly participate in metal coordination. ICP-MS data further indicate that both sulfur atoms from C170 and C171 are required for tight Ni-binding, as neither sulfur atom alone appears to be sufficient.

The spectral features generated by PRL-1 also suggest Ni is coordinated by nitrogen atoms in the remaining two positions. In model proteins that bind Ni using square planar geometry, some of the ligating nitrogen groups become deprotonated, and the negatively charged species binds the metal. The absorption band at 526 nm indicates that PRL-1 coordinates Ni via a deprotonated nitrogen, whereas the 421 nm absorption generated in the PRL-1 spectrum likely reflects coordination by a singly protonated nitrogen. Mutations of the His in the GGH-like motif to make PRL-1-H166A revealed that this histidine's side chain does not substantially influence Ni binding. PRL-1 encodes three other His in addition to H166. H23, H64 and H103 are located in distant regions of the sequence and are unlikely to remain tightly associated under denaturing conditions. Attempts to unfold the protein so as to release the metal required vigorous treatment with high concentrations of denaturants at high temperature for several hours. As chemical and heat denaturation permit only slow release of Ni and CD data show that PRL-1 completely unfolds well before Ni is released, the metal is coordinated by atoms clustered in a short segment of the protein.

The GGH-type peptides achieve extremely tight binding (K_{d}˜10⁻¹⁷-10⁻¹⁸), which is accomplished in part by deprotonated backbone amide nitrogens within the motif. In light of the tight binding observed for PRL-1 and the predicted contribution from nitrogen atoms to Ni ligation, backbone amides from PRL-1 may participate in metal coordination. Because the absence of either cysteine abolishes binding and truncation removes both the side chain and the backbone amide, testing the hypothesis that metallation involves the amide nitrogens adjacent to C170 and C171 using mutagenesis is not feasible.

### Example 7

Cu-NCC at physiological pH was placed in a tube surrounded by saline bags and imaged using a standard MRI at the Hoglund Brain Imaging Center at the University of Kansas Medical Center (FIG. 14). In FIG. 14, the bright signal in the center corresponds to the Cu-NCC complex, whereas the less bright signal corresponds to the saline solutions. Black corresponds to the tube and air.

### Example 8

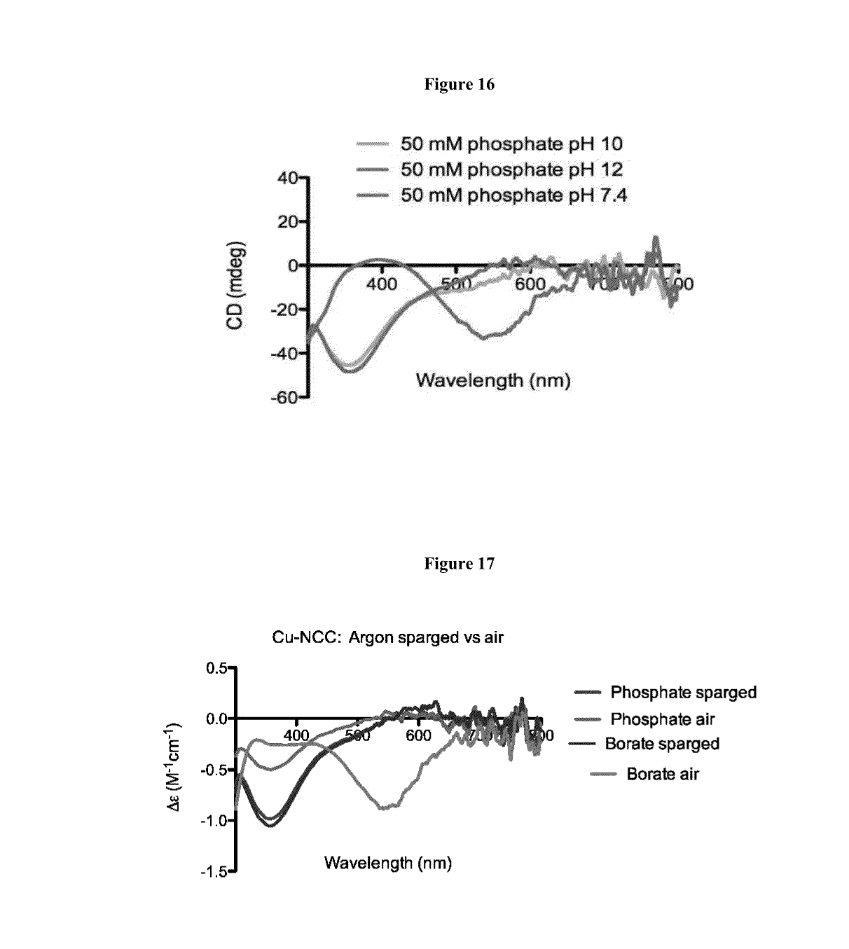

Initial work with Cu-NCC indicated a lack of stability of the complex. Freezing the Cu-peptide complex caused it to change color. Over time it would lose color or change color. Generally, the Cu complex was a brownish/orangish color with a hint of green that changed to a pink species over a period of minutes-hours; however, in some conditions, the pink species would be generated immediately (i.e. at higher pH, without Ar sparging). In all cases, the complex would eventually turn pale pink and later clear. The CD profiles of each of these species were different (in FIG. 16, the pH 12 species is of the pink-colored sample).

The pink species was more readily generated (faster, if not immediately) in the presence of oxygen. At pH 10 in borate and air, it immediately became the pink species, whereas in phosphate, the pink species formed more slowly over time. At the same two pH values when the samples were sparged, the two behaved identically to each other. Therefore, the pH and oxygen content affect the rate of cleavage. See FIG. 17.

Copper II is most often blue/green (6-coordinate) or orange/brown (4-coordinate), whereas copper I is typically pink (2-coordinate) in color. The reaction was performed using Cu(II), and therefore, the orange/brown color reflects the formation of a 4-coordinate Cu(II) complex. The change in color to a pink species suggests that there is a change in structure and/or metal coordination.

Analysis of NCC Embedded in Longer Peptide Sequences

Copper Complexes with 5mers (Pentapeptides): C-Terminal Placement of NCC.

Cu-5mer complexes were attempted at pH 7.4 to transmetallate the pentapeptides GGNCC (SEQ ID NO: 26), GGGCC (SEQ ID NO: 27), GNNCC (SEQ ID NO: 28), and GNGCC (SEQ ID NO: 29) with copper charged IMAC as per our standard protocol; however, a change in color was not observed with the eye or UV/vis absorption spectroscopy and metal incorporation could not be detected. Cu binding to NCC was, however, observed in the full-length phosphatase of regenerating liver (PRL-1) protein. This suggested that copper was capable of binding when NCC was embedded in longer sequences. These studies suggested that the peptide was undergoing cleavage in response to Cu coordination and that it was happening so quickly that immediately following coordination the peptide instantly underwent the cleavage reaction such that a color change could not be observed; it was essentially cutting up the metal binding site before the complex could be observed.

Copper Complexes with 8mers (Octapeptides): Internal Placement of NCC within Longer Peptides.

Octameric peptides were tested to determine if there was specificity in the cleavage that was occurring: GGNCCGGK (SEQ ID NO: 30), GGGCCGGK (SEQ ID NO: 31), GGNCHGGK (SEQ ID NO: 32). CD and absorbance were used to examine this and observe spectral profiles for each. MS was used to detect intact peptide, Cu complexes with the peptide, and any peptide fragments generated that were large enough to be observed by MS.

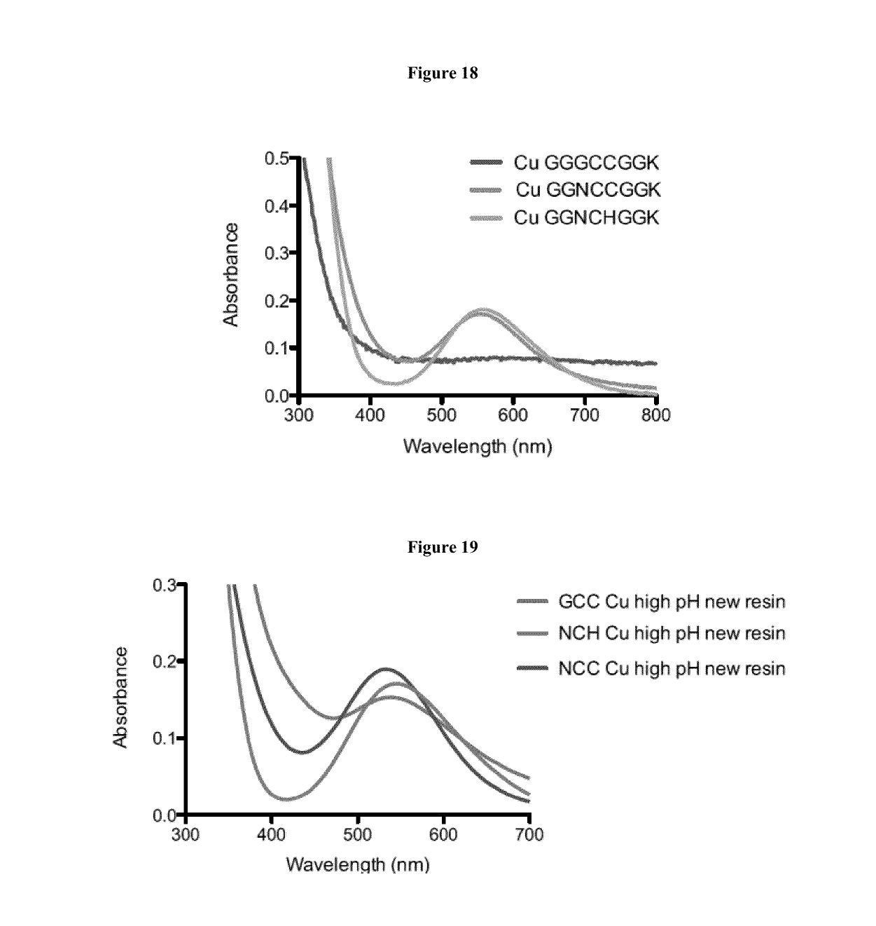

CD and Abs of copper peptides at pH 7.4 that had been transmetallated with Cu-charged IMAC resin under different conditions revealed some differences in the reaction. Under Ar-sparged conditions (pH 7.4, phosphate buffer) GGGCCGGK (SEQ ID NO: 31) peptide did not change color upon incubation with the resin, but the other two 8mer peptides changed color. The presence of the asparagine likely changed the kinetics of the cleavage reaction, suggesting altering the amino acid composition at the first position in the NCC sequence can be used to tune the cleavage rate. See FIG. 18.

If the reaction was performed at higher pH (−8.5), the GGGCCGGK (SEQ ID NO: 31) species was brownish/yellowish and the intact metal-8mer complex was detectable using MS. The other 2 peptides immediately formed a pink species and were not observable by MS in their intact form. This suggested that not only the sequence but the incorporation conditions (pH and buffer) affect the kinetics of the cleavage reaction. See FIG. 19.

The resin used to transmetallate the species retained a range of different colors following incubation with the peptide under some conditions. While the GCC-8mer resin remained blue, the NCC-8mer resin was a darker blue and the NCH-8mer resin was purplish.

Peptide Cleavage

ESI-MS showed that the 8mer peptides fragment after complexation with copper. The table below shows the expected and observed mass fragments: Indicating that cleavage readily occurred on the C-terminal side of the peptide cleavage tag sequence.

TABLE 1
Peptide fragment masses

| GGNCCGGK(694.8)(SEQ ID NO: 30) | mass |
|---|---|
| GG | 132* |
| GGN | 246.2 |
| GGNC (SEQ ID NO: 32) | 349.4 |
| GGNCC (SEQ ID NO: 26) | 452.5 |
| NCCGGK (SEQ ID NO: 33) | 580.7 |
| CCGGK (SEQ ID NO: 34) | 466.6 |
| CGGK (SEQ ID NO: 35) | 363.4 |
| GGK | 260.3 |
| GGGCCGGK(637.7)(SEQ ID NO: 31) | mass |
| GG | 132* |
| GGG | 189* |
| GGGC (SEQ ID NO: 36) | 292.3 |
| GGGCC (SEQ ID NO: 27) | 395.5 |
| GCCGGK (SEQ ID NO: 37) | 523.6 |
| CCGGK (SEQ ID NO: 38) | 466.6 |
| CGGK (SEQ ID NO: 39) | 363.4 |
| GGK | 260.3 |

KEY: *Fragment is too small to observe with MS

Similar changes in color were observed with the use of iron and as such, iron binding may also lead to peptide cleavage.

### Example 9

### Generation of Metal Peptide Complexes

The peptides NCC, GCC, and NCC with a D-cysteine in the middle position [LDL-NCC] were purchased from Genscript Corporation (Piscatway, N.J., USA). The NCC peptides with a D-cysteine in the third position [LLD-NCC] and with both D-asparagine in the first position and D-cysteine in the third posistion [DLD-NCC] were purchased from Neo-Peptide (Cambridge, Mass., USA). Nickel-peptide complexes were generated in aqueous solution at neutral to basic pH. Incubation of the peptide for approximately 30 minutes with immobilized metal affinity chromatography (IMAC) resin (GE Healthcare) charged with nickel ensured a clean reaction with no undesired side products and no free metal ions in solution, yielding a reddish-brown complex, varying slightly by the identity of the peptide. Aged Ni-NCC samples were allowed to age for >40 days.

CD and Absorption Studies

A 1.5 mM solution of Ni-NCC was prepared in 50 mM potassium phosphate, pH 7.4, and used as is or sparged with argon. Immediately after incubation, samples were placed in a cuvette with a 1-cm pathlength and scanned from 800-300 nm using both absorption and CD spectroscopy. Samples were aged and monitored at various timepoints over the course of several days. Background scans of buffer alone were subtracted from each scan. Absorption studies were performed on an Agilent 8453 UV/Visible spectrophotometer. Circular dichroism analysis was performed on a J-815 (Jasco Corporation) spectrapolorimeter. The CD data presented represent the average of at least five scans. To accurately control the time frame of Ni-NCC aging for activity assays, the complex also was formed in solution upon addition of one equivalent of NiSO₄.

MCD Experiments

Samples of Ni-NCC were prepared in 50 mM potassium phosphate buffer at pH 7.4 sparged with argon. Solid sucrose was added as a glassing agent and the mixture was heated to form a saturated solution. CD spectra of sucrose-saturated samples demonstrated no significant changes in features compared with samples lacking sucrose, indicating that this procedure did not perturb the structures of the Ni-NCC complex. The samples were placed in an MCD cell and flash frozen in liquid N₂. Spectra were collected on a J-815 (Jasco Corporation) spectropolarimeter interfaced with a magnetocryostat (Oxford Spectromag 4000-8). To remove contributions from CD signals, MCD data represent difference spectra of accumulations at +7 and −7T. Because the signal intensities from paramagnetic species display inverse temperature dependence, spectra were collected at several temperatures.

Deconvolution of CD and Absorption Data:

Deconvolution of CD and absorption data was performed using Igor Pro (Wavemetrics). Iterative Gaussian deconvolutions were performed with a constant peak width of 1650 cm. Absorption band energies were kept within 10% of the corresponding CD bands due to the broad nature of the absorption spectrum.

ESI-MS:

Samples were diluted 100× in a 1:1 mixture of methanol/water and analyzed on an LCT Premier (Waters Corporation) operating in negative ion mode, as described previously.

Deuterium Exchange:

A solution of 50 mM potassium phosphate was prepared in D₂O and adjusted with NaOD and DC1 to a pD of 7.4. Samples of 1.5 mM NCC and GCC were prepared in this solution. Transmetallation was performed as described. After removal of the solid resin, 10 μL of the sample was back-exchanged into one mL of a 1:1 water/methanol mixture and analyzed using ESI-MS operating in negative ion mode. The original samples were then incubated for 24 hours and analyzed in the same manner.

Electrochemistry:

Electrochemical data were collected. A 3 mL sample of 3 mM Ni-NCC was prepared in 50 mM sodium borate at pH 10. CV data were collected with a CH1812C Electrochemical Analyzer potentiostat (CH Instruments) with a three-electrode setup (platinum working electrode, Bioanalytical Systems, Inc.; Pt auxiliary electrode; Ag/AgCl reference electrode) in a glass CV cell. Potential was applied from zero to 1.2 V with a scan rate of 0.2 V per second, and current was measured. The same experiment was attempted on a 3 mM Ni-NCC sample in 50 mM potassium phosphate at the same pH. The sample was incubated for 24 hours and analyzed again.

Coordination of Cyanide and IR Analysis:

Samples of Ni-NCC were prepared at a concentration of 3 mM in 50 mM sodium borate at pH 10 and 50 mM potassium phosphate at pH 7.4. A sample of Ni-NCC in phosphate buffer was incubated for 24 hours. One equivalent of potassium cyanide was added to each of the three Ni-NCC samples. Samples were flash frozen and lyophilized. IR analysis was performed to observe the cyanide peak in each sample. IR spectra were acquired from dry powder samples on a Perkin Elmer Spectrum 100 FT-IR spectrometer equiped with a universal ATR (Attenuated Total Reflection) sampling accessory. The spectrum of solid potassium cyanide was used to compare the shift of v(C≡N) vibration from the free to the nickel-coordinated state.

Computations:

Spin-restricted density functional theory (DFT) computations were performed using ORCA 2.8.0 and employed the conductor-like screening model (COSMO) with an epsilon value of 80 to approximate water. Geometry optimizations used the BP86 functional and the aug-TZVP basis set (a triple-zeta basis set with diffuse and polarization functions). Because these computations employed the resolution of identity (RI) approximation, the TZV/J auxiliary basis set was also used. Single point and time-dependent DFT (TD-DFT) computations used the B3LYP functional and the aug-TZVP basis set. In order to evaluate if the inclusion of explicit water molecules H-bonded to charged groups gave rise to geometries markedly different than those obtained using COSMO, a water molecule was added to hydrogen bond with the C-terminal carboxylate in the LLL, DLD, and DDL models of Ni-NCC. Because differences in bond lengths of less than 0.015 \acute{Å} were observed, we reasonably conclude that the use of COSMO is sufficient to account for major solvation effects in this system.

Ni-SOD xanthine/xanthine oxidase coupled assay: Ni-SOD activity was determined, except Ni-NCC was generated in situ using one equivalent NiSO₄. Ni-NCC was aged for 0-120 minutes, and the Ni-SOD activity was determined using the standard xanthine/xanthine oxidase method developed by Crappo and coworkers. All reagents were generated in 50 mM potassium phosphate, 100 μM EDTA reaction buffer at pH 7.8 except for Ni-NCC, which was generated in 50 mM potassium phosphate, pH 7.4. 600 μM cytochrome c from bovine heart (Sigma), 300 μM xanthine (Sigma) and enough xanthine oxidase from buttermilk (Sigma) to cause a change in absorbance at 550 nM of 0.02-0.04 AU per minute were added to a final volume of 300 μL, with reaction buffer. The change in absorbance at 550 nm was monitored on a Cary 100 UV-Visible spectrophotometer (Varian). The assay was performed with 100 μM Ni-NCC.

Preparation and Spectroscopic Characterization of Ni-NCC:

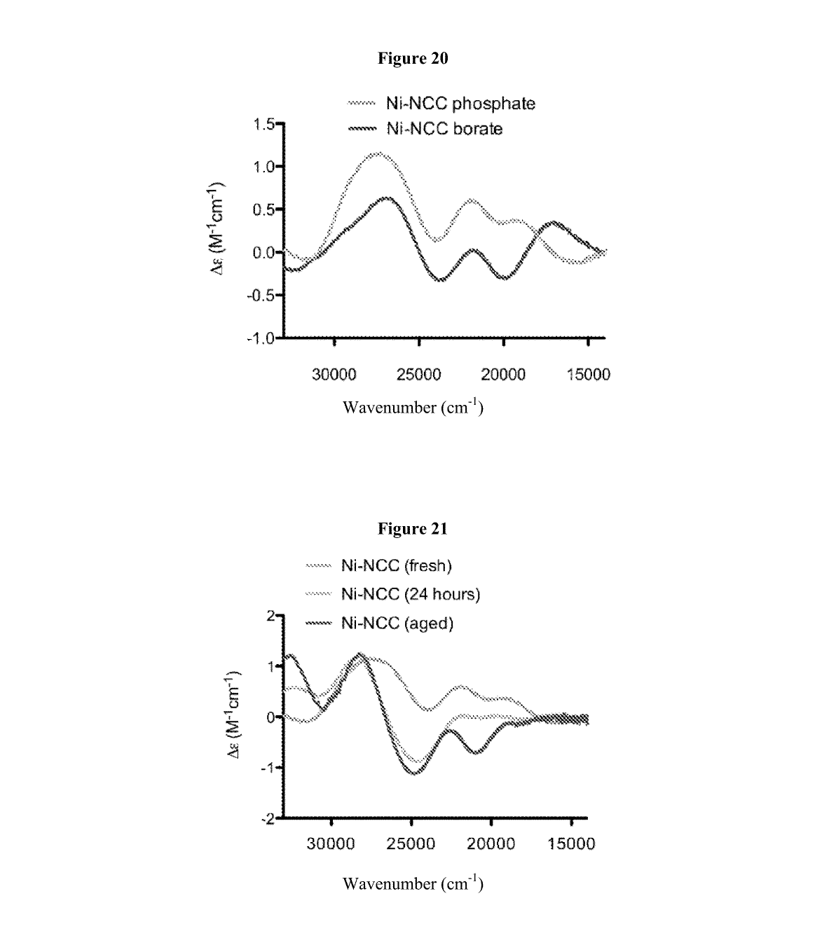

Metal incorporation into the described complex was accomplished via transmetallation. Peptides were incubated with IMAC resin in either 50 mM potassium phosphate buffer at pH 7.4 or in 50 mM sodium borate at pH 10. Absorption and CD spectroscopies were utilized to characterize the Ni-NCC complex. Studies have shown that pH, ionic strength, and concentration of the metal-peptide complex do not change the spectral features of the system; however, these studies show that the initial spectra differ between buffer systems (FIG. 20) but later converge to a common final state. pH was not a factor in the differences between spectra, as Ni-NCC samples analyzed in phosphate buffer at pH 10 directly after incubation exhibited the same spectral features as those in phosphate buffer at pH 7.4.

Over time, however, the spectral features of Ni-NCC in phosphate buffer changed to resemble those in borate buffer, suggesting rearrangement to a more stable structure occurred. Although samples were prepared in sparged solutions, no further precautions were taken to avoid oxygen dissolution during aging. To ensure complete conversion, a Ni-NCC sample in phosphate buffer was aged in air for up to 90 days and analyzed again (FIG. 21). ESI-MS of the Ni-NCC complex in various conditions demonstrated that the peptide mass did not change with the changes in spectral features (m/z=392.98), indicating a lack of oxidation of thiolate ligands. Varied pH did not affect the overall rate of the aging process. This suggests that the changes observed in the CD are due to more subtle changes about the metal center. A Ni-GCC sample was examined in the same manner to evaluate the influence of the chirality at the first position. Although the spectral changes for the two complexes are not identical, a similar perturbation of CD signals was observed for Ni-GCC over a comparable time frame.

Spectral Deconvolutions:

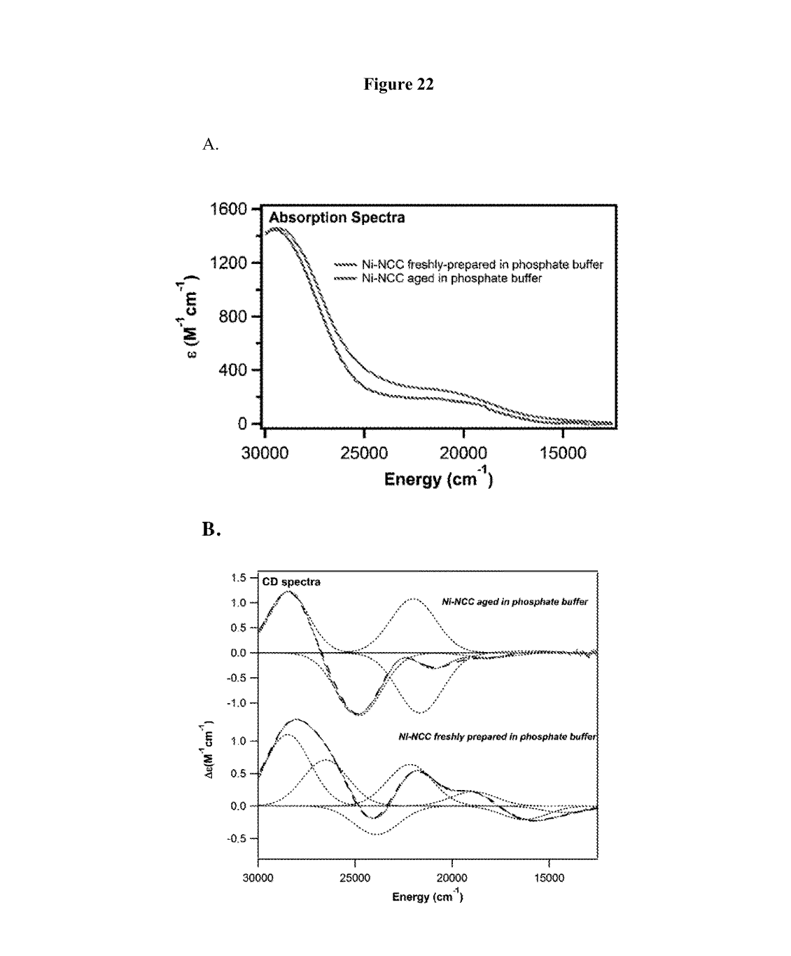

In contrast to the changes in the CD spectra of Ni-NCC, absorption spectra of Ni-NCC freshly prepared in phosphate buffer and that same sample aged for 40 days appeared nearly identical (FIG. 22A). Both feature a broad envelope centered at 21 000 cm⁻¹ (ε=210 M⁻¹cm⁻¹) with a higher energy feature at 29 000 cm⁻¹ (ε=1400⁻¹ cm⁻¹). These data are consistent with both species having a four-coordinate Ni^{II} center in an N₂S₂ square planar geometry. To quantitatively evaluate the CD spectral changes, spectral deconvolutions of these data were performed to determine the energies and signs of the electronic transitions (FIG. 22B). Whereas freshly prepared Ni-NCC displays signals with positive sign at 18 900, 22 170, and 26 520 cm¹, the spectrum of the aged sample shows negative bands at similar energies (Table 2). These transitions shift to slightly lower energies (Δ˜200-900 cm⁻¹) with age. Bands 1-4 of freshly prepared Ni-NCC were previously assigned as d-d transitions) and their energies should be very sensitive to changes in geometry about the Ni^{II} center. The observation that the energies of these bands shift by <900 cm⁻¹ upon aging demonstrates that fresh and aged Ni-NCC have nearly identical coordination environments. The major spectral perturbations are predominately due to changes in sign and intensity of CD features. These minor changes in the d-d transition energies (Table 2) along with the virtually identical absorption spectra (FIG. 22A) of fresh and aged Ni-NCC demonstrate that the geometry and ligands of the Ni^{II} center are unaltered and that neither dimerization of the complex nor oxidation of the thiolate ligand occurs.

TABLE 2
Transition energies derived from Gaussian deconvoluted
CD spectra of freshly prepared and aged Ni-NCC in
phosphate buffer. Bands having opposite sign in the
fresh and aged spectra are shown in italics.

| | |
|---|---|
| Freshly Prepared | Aged |

| | Δε | Energy | Δε | Energy |
|---|---|---|---|---|
| Band | (M⁻¹ cm⁻¹)⁻¹ | (cm⁻¹) | (M⁻¹ cm⁻¹) | (cm⁻¹) |
| 1 | −0.1 | 14200 | ^{a} | ^{a} |
| 2 | −0.21 | 16270 | 0.04 | 16000 |
| 3 | 0.22 | 18900 | −0.1 | 18000 |
| 4 | 0.64 | 22170 | −0.78 | 21600 |
| 5 | −0.44 | 23900 | 0.7 | 22000 |
| 6 | 0.71 | 26520 | −1.3 | 24800 |
| 7 | 1.1 | 28475 | 1.22 | 28450 |

^{a} The low signal-to-noise ratio between 11 000 and 14 000 cm′ precludes reliable deconvolution within this spectral window.

Magnetic Circular Dichroism:

To investigate whether a paramagnetic, tetrahedral intermediate is formed, magnetic CD (MCD) experiments were performed. Previous MCD experiments have shown that the primary Ni-NCC species in borate buffer at pH 10 is largely diamagnetic, although a minor paramagnetic (S=1) species was present. Here, MCD experiments performed on a freshly prepared Ni-NCC sample demonstrated that the species initially present in phosphate buffer is also primarily diamagnetic; a minor paramagnetic component that accounts for less than 1% of the sample may reflect an intermediate state that does not accumulate. After aging, these temperature-dependent signals are no longer observed. Therefore, it can be concluded that aged Ni-NCC contains neither appreciable amounts of S=1 Ni^{II} or S=1/2 Ni^{II} centers. These data collectively show that both fresh and aged Ni-NCC contain diamagnetic Ni^{II} centers. These data collectively show that both fresh and aged Ni-NCC contain diamagnetic Ni^{II} in square planar geometries with nearly identical coordination spheres; the differences in spectral features and in reactivity between the freshly prepared and aged samples led to further investigation of the subtle changes in the Ni-NCC complex.

Electrochemistry and Reactivity of Ni-NCC.

Electrochemical experiments have shown that Ni-NCC in borate buffer at pH 9.3 has a midpoint potential of 0.72 V (vs. Ag/Ag⁺). When attempts to measure the midpoint potential of Ni-NCC in phosphate buffer at the same pH were made, the complex prepared in phosphate buffer did not exhibit a measurable potential; however, when the same sample was aged for 24 hours, the midpoint potential was comparable to that of the sample in borate buffer (0.71 V vs. Ag/Ag⁺). Previous studies have shown that square planar geometries, for example peptide mimics of Ni-SOD, coordinate cyanide in an axial position, as determined by IR of the bound cyanide. Similarly, IR experiments have demonstrated that Ni-NCC in borate buffer is capable of coordinating CN in the axial position, as a shift in the v (C≡N) vibration occurs. The IR spectrum of Ni-NCC freshly prepared in phosphate buffer did not exhibit a peak corresponding to coordinated cyanide; however, addition of cyanide after aging the sample overnight generated the expected peak for the coordinated state (Table 3). These data lend support to a slow structural rearrangement that occurs over the course of hours, resulting in a structure that allows for the interaction of a fifth ligand with the Ni-NCC complex.

TABLE 3
IR data for cyanide coordinated to nickel.

| Species | ν(C≡N) (cm⁻¹) |
|---|---|
| NaCN | 2088 |
| K₂[Ni(CN)₄]⁴² | 2123 |
| Ni(CN)-(mSOD)⁴² | 2108 |
| Ni-NCC + CN borate | 2109 |
| Ni-NCC + CN | N/A |
| phosphate (fresh) | |
| Ni-NCC + CN | 2107 |
| phosphate (aged) | |

Deuterium Exchange:

The flip in sign of CD signals suggests the structural change that allows for ligand binding may be due to chiral inversion. Because of this possibility, NCC was transmetallated with Ni-IMAC resin in buffers prepared in D₂O to determine if deuterium would be incorporated into the peptide at any non-exchangeable site. After back-exchanging the Ni-NCC into 1:1 water/methanol to preserve the integrity of the complex but remove any exchangeable deuterium atoms, ESI-MS demonstrated incorporation of deuterium into two non-exchangeable positions (392.98 vs. 394.99). ESI-MS of the same reaction performed in H₂O showed no difference in m/z over 24 hours. Because Ni-GCC shows a similar inversion of CD signals with time, but lacks chirality in the first position, Ni-GCC was also examined for deuterium exchange. Ni-GCC exhibited incorporation of deuterium into one non-exchangeable position, suggesting that the chirality of Asn in the first position and only one of the Cys is affected. This information was used to predict the possible location(s) of the incorporated deuterium atom(s).

Characterization of Peptides Containing D Amino Acids:

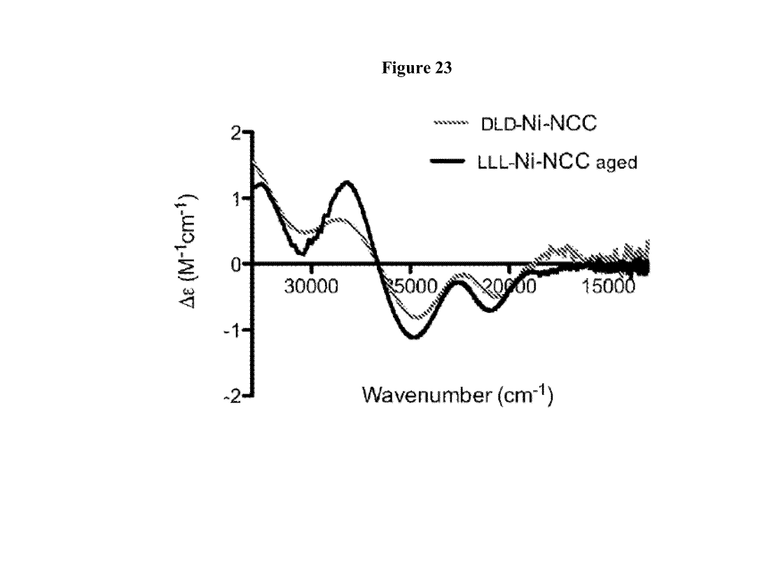

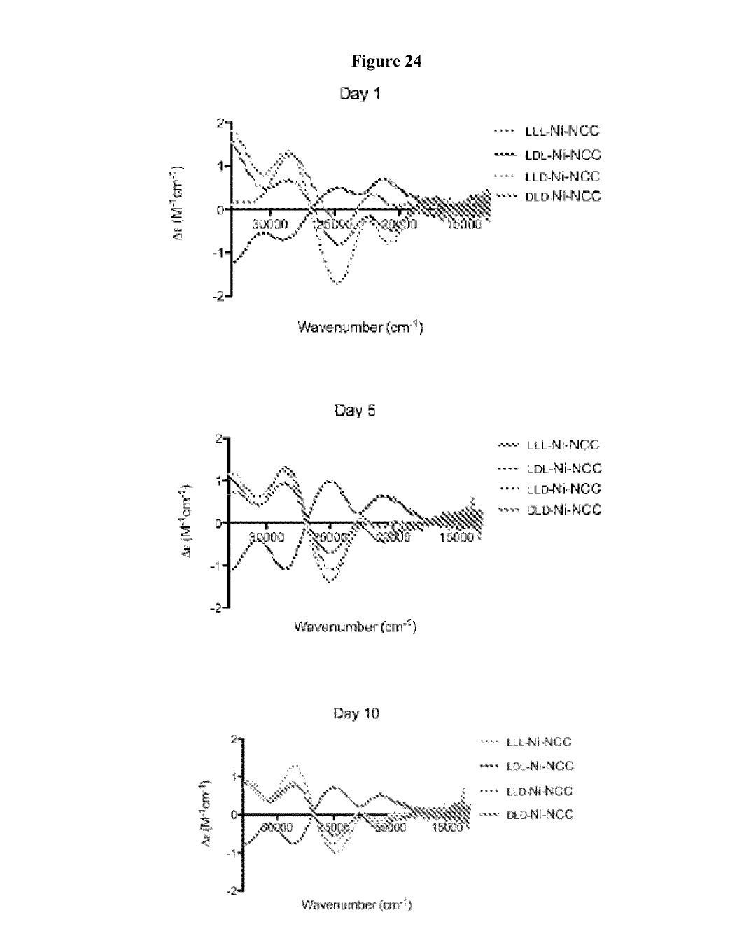

The LDL-Ni-NCC, LLD-Ni-NCC, and DLD-Ni-NCC complexes were generated and each analyzed using absorption and CD spectroscopies and ESI-MS. All of the complexes exhibit the same mass profile (m/z=392.98) in ESI-MS, suggesting that each forms 1:1 complexes with the metal. To determine the chirality of the final, stable Ni-NCC arrangement, CD spectra of each D-containing peptide were compared to the data collected for the aged (>40 days) Ni-NCC sample in phosphate buffer. The DLD-Ni-NCC spectrum overlaid with the aged Ni-NCC spectrum shows parallel features (FIG. 23). These data indicate chiral inversion occurs at the first and third position within Ni-NCC to generate the DLD-Ni-NCC complex.

Whereas the spectral features of DLD-Ni-NCC do not shift or lose intensity with time, the CD spectrum of LLD-Ni-NCC evolves over time to look like that of DLD-Ni-NCC (FIG. 24). In contrast, the CD spectrum of aged LDL-Ni-NCC looks like the mirror image of that of DLD-Ni-NCC. When the freshly prepared peptides were reacted with cyanide, only DLD-Ni-NCC was able to immediately coordinate cyanide in the axial position, providing further evidence that the DLD-form is the arrangement that aged Ni-NCC reaches over time.

DFT-Optimized Models and Computed Energies:

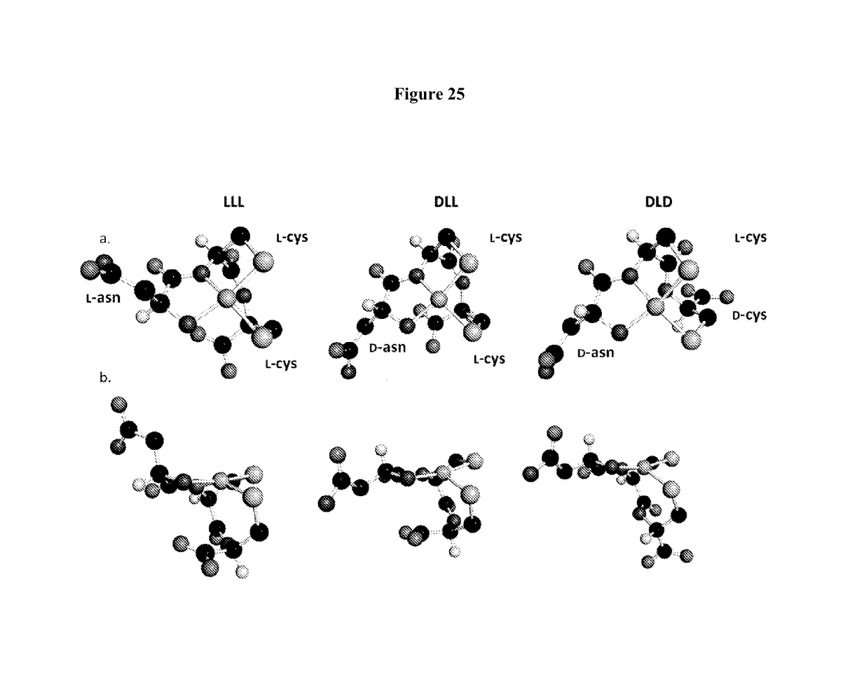

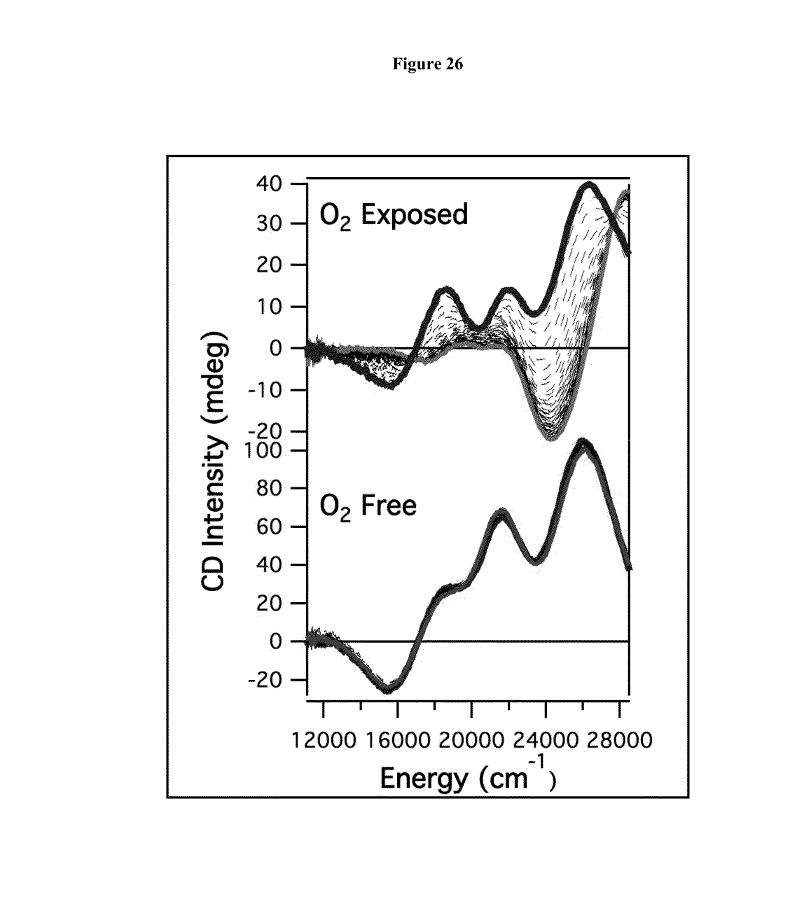

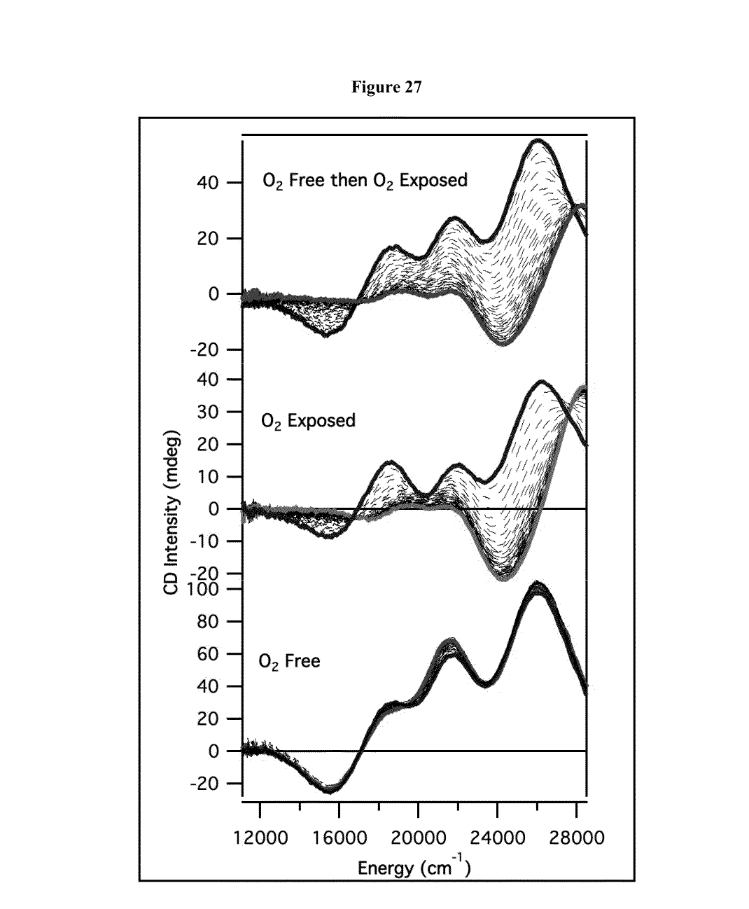

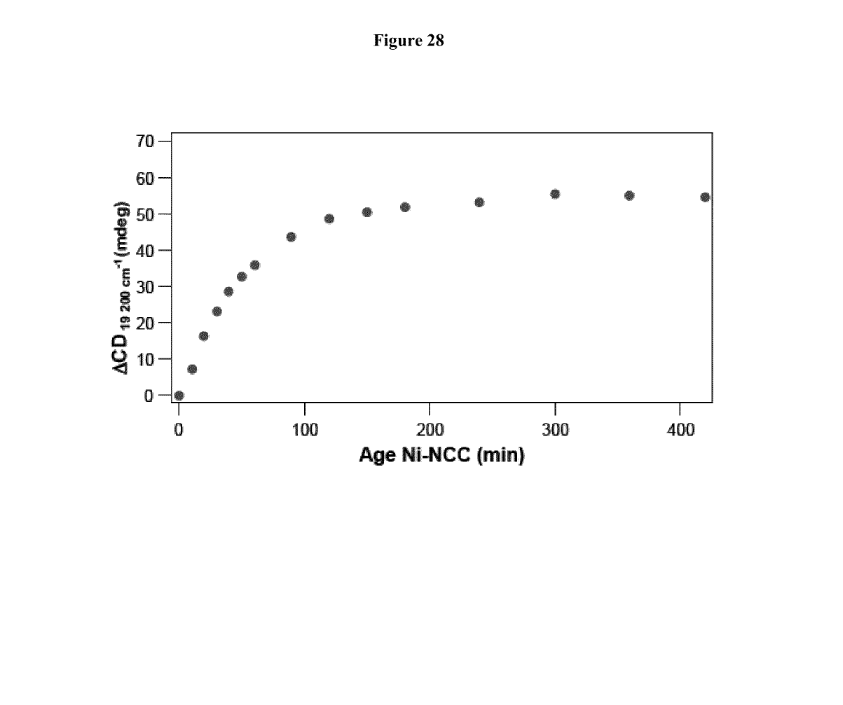

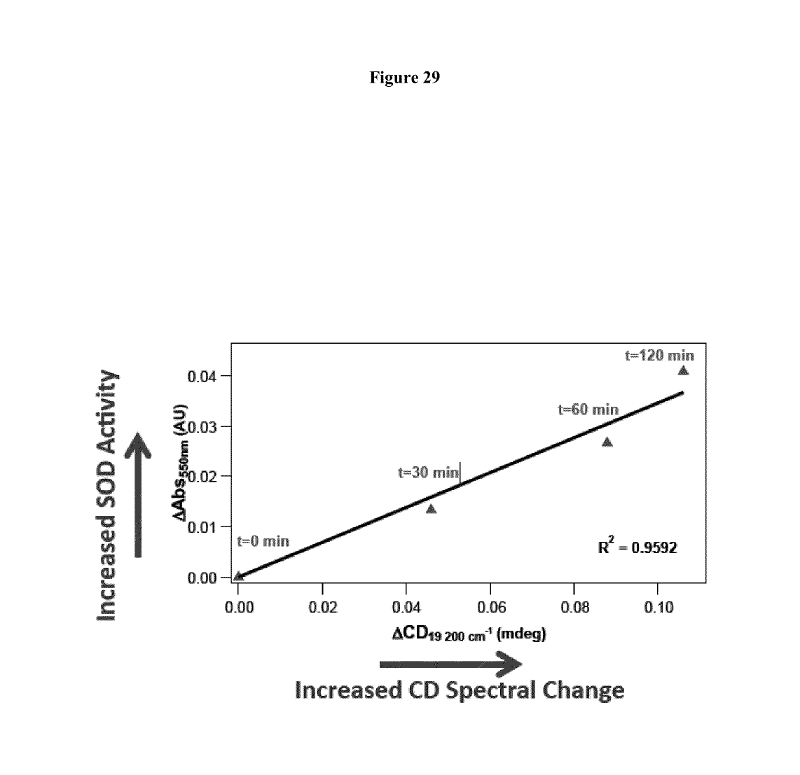

Computations were performed on models of Ni-NCC to explore the structural and energetic changes associated with chiral inversion of the different amino acid residues in the tripeptide-Ni^{II} complex. These results are summarized in Table 4. In the optimized structure of LLL-Ni-NCC, the nickel(II) ion is bound in a near-square planar geometry (FIG. 25). The coordination of the terminal amine, internal amide, and sulfur of Cys2 form two five-membered chelate rings that share a common edge. The sulfur of Cys3 coordinates trans to the amide nitrogen, which requires that the peptide wrap around the nickel center, thereby blocking one coordination site perpendicular to the square plane. The other open coordination site is partially blocked by the Asn side chain. These results suggest that the metal center may be sterically occluded in the LLL-peptide complex, explaining the lack of CN coordination observed in the freshly prepared samples described above.

The energies of Ni-NCC models with the chirality of different amino acids inverted show that inversion of Asn 1 leads to a model (DLL-Ni-NCC) that is isoenergetic to that of LLL-Ni-NCC. In addition, the models in which Cys2 is inverted, LDL- and DDL-Ni-NCC, have an energetic destabilization of ˜5 kcal/mol when compared to LLL-Ni-NCC. In contrast, when Cys3 is inverted, as in LLD- and DLD-Ni-NCC, an energetic stabilization of ˜11 kcal/mol is predicted. Investigation into the role of solvation on the total energy of the conformers shows that although DLD-Ni-NCC does have an overall stabilization with respect to LLL-Ni-NCC in solvation energy, the relief of steric strain of the loop containing Cys3 has a more pronounced effect on the total energy. These results for DLD-Ni-NCC agree with the experimental data described above (FIG. 23). The inversion from LLL-Ni-NCC to DLD-Ni-NCC opens one face of the Ni^{II} ion to interact with exogenous ligands, consistent with the observation that aged Ni-NCC binds CN.

TABLE 4
Bond lengths (Å) and relative energies (kcal/mol) of DFT-optimized
models of Ni^{II}-NCC with amino acids of differing chiralities.

| | LLL-Ni-NCC | DLL-Ni-NCC | LDL-Ni-NCC | LLD-Ni-NCC | DDL-Ni-NCC | DLD-Ni-NCC |
|---|---|---|---|---|---|---|
| Ni-Nₐₘᵢₙₑ | 1.976 | 1.972 | 1.974 | 1.982 | 1.979 | 1.990 |
| Ni-Nₐₘᵢₙₑ | 1.890 | 1.856 | 1.867 | 1.866 | 1.866 | 1.887 |
| Ni-S_{cys2} | 2.222 | 2.184 | 2.165 | 2.179 | 2.164 | 2.196 |
| Ni-S_{cys3} | 2.161 | 2.199 | 2.199 | 2.221 | 2.199 | 2.238 |
| Relative | 0.0 | −0.3 | 4.7 | −11.6 | 5.1 | −11.1 |
| Energy | | | | | | |

TD-DFT Computations:

TD-DFT computations were performed to determine if the structural differences between LLL-Ni-NCC and DLD-Ni-NCC can account for the experimentally observed red-shift in the Ni^{II} d-d transition energies upon aging of Ni-NCC. Because Ni—S bond lengths are frequently overestimated in DFT geometry-optimized models, it is expected that the predicted electronic transition energies for both Ni-NCC models will be computed at lower energy than experimentally observed. Nonetheless, this known shortcoming in DFT-computed Ni—S bond lengths will not hinder the analysis performed here, as the focus is on reproducing the relative shift in Ni^{II} d-d transitions between LLL-Ni-NCC and DLD-Ni-NCC. For an S=0, d⁸ metal ion in a square planar geometry, four d-d transitions are expected from excitation from each of the four doubly-occupied d orbitals to the unoccupied dₓ²_{-y}² orbital. The computed relative energies of these transitions are shown in Table 4. Relative to LLL-Ni-NCC, all calculated d-d transitions for DLD-Ni-NCC are red-shifted, consistent with the experimental observation. This shift in transition energies is directly related to the energy of the dₓ²_{-y}² orbital. In an idealized square planar geometry (e.g., D₄ₕ symmetry), the dₓ²_{-y}² orbital is the dominant σ* orbital and is significantly destabilized relative to the remaining four d orbitals. As the geometry is perturbed from this limit, other d orbitals take on partial σ* character. This leads to the dₓ²_{-y}² orbital being at a relatively lower energy and a red shift in d-d transitions. The nickel(II) coordination sphere in DLD-Ni-NCC is more distorted from square planar geometry than that of LLL-Ni-NCC, which gives rise to a smaller splitting between the Ni^{II} d orbitals and thus red shifted d-d transitions.

Studies performed in the absence of oxygen result in metal binding but chiral inversion does not occur. Introduction of oxygen into the Ni-NCC sample facilitated conversion to the DLD state. Therefore in addition to the metal being bound in the unique MAP configuration, an electron transfer agent, in this case derived from molecular oxygen, must be present in various embodiments for modulation of the peptide. The concentration or partial pressure of oxygen may be altered to alter the rate of the reaction.

While preferred embodiments of the present invention have been shown and described herein, such embodiments are provided by way of example only. Numerous variations, changes, and substitutions may be made without departing from the invention. It should be understood that various alternatives to the embodiments of the invention described herein may be employed in practicing the invention.

1. A method of performing a site-specific chiral inversion of a tripeptide comprising complexing a metal ion with a tripeptide having a sequence LXLC₁LC₂ to form a metal ion-DXLC₁DC₂ complex,
wherein X is any amino acid such that the tripeptide and metal ion form a complex having a square planar orientation or square pyramidal orientation or both, and wherein C₁ and C₂ are the same or different; and wherein C₁ is chosen from a cysteine and a cysteine-like nonnatural amino acid; and wherein C₂ is chosen from a cysteine, a cysteine-like nonnatural amino acid, a histidine, and a histidine-like nonnatural amino acid.

2. (canceled)

3. The method of claim 1, wherein the metal ion is selected from the group consisting of nickel and palladium.

4. The method of claim 3, wherein the metal ion is nickel.

5. The method according to claim 1, wherein LX and DX are glycine.

6. (canceled)

7. (canceled)

8. (canceled)

9. A method of providing anti-oxidant properties to a composition comprising
(i) providing a plurality of amino acids having a sequence LXLC₁LC₂ and a metal ion in a composition; and
(ii) performing the method of claim 1.

10. An anti-oxidant composition comprising a metal ion complexed with a tripeptide having the sequence LXLC₁LC₂ to form a metal ion-DXLC₁DC₂ complex;
wherein X is any amino acid such that the tripeptide and metal ion form a complex having a square planar orientation or square pyramidal orientation or both, and wherein C₁ and C₂ are the same or different; and wherein C₁ is chosen from a cysteine and a cysteine-like nonnatural amino acid; and wherein C₂ is chosen from a cysteine, a cysteine-like nonnatural amino acid, a histidine, and a histidine-like nonnatural amino acid.

11. A method of performing a site-specific cleavage of a peptide comprising:
providing a peptide with a sequence XC₁C₂, wherein X is any amino acid such that the peptide and metal ion form a complex having a square planar orientation or square pyramidal orientation or both, and wherein C_{I} and C₂ are the same or different; and wherein C₁ is chosen from a cysteine and a cysteine-like nonnatural amino acid; and wherein C₂ is chosen from a cysteine, a cysteine-like nonnatural amino acid, a histidine, and a histidine-like nonnatural amino acid; and
combining a partial chelator with a metal capable of reacting with the sequence XC₁C₂; and
allowing cleavage of the peptide within or at a terminal portion of the sequence XC₁C₂.

12. The method of claim 11, wherein the metal is copper or iron.

13. The method of claim 11, further comprising an electron transfer component.

14. The method of claim 13, wherein said electron transfer component is selected from O₂ or a species donating an electron.

15. The method of claim 11, wherein the peptide further comprises at least one selected from the group consisting of: an affinity tag, a peptide, and a protein.

16. The method of claim 11, wherein the peptide further comprises an affinity tag, the method further comprising immobilizing the peptide on a substrate for the affinity tag; and releasing a portion of the peptide from the substrate.

17. The method of claim 16, further comprising recovering a portion of the peptide.

18. (canceled)

19. (canceled)

20. A kit comprising a peptide having a sequence comprising XC₁C₂, wherein X is any amino acid, C₁ and C₂ are the same or different, C₁ is chosen from a cysteine and a cysteine-like nonnatural amino acid, and C₂ is chosen from a cysteine, a cysteine-like nonnatural amino acid, a histidine, and a histidine-like nonnatural amino acid; and a partial chelator with a metal capable of reacting with the sequence XC₁C₂.

21. A method of protein purification comprising;
a) binding a first composition comprising a target protein associated with a linker and an affinity tag to an affinity material comprising a ligand capable of capturing the first composition;
b) providing a metal-containing cleavage agent capable of detaching the target protein from the first composition such that the detached target protein is devoid of any supplementary amino acids originating from the linker or the affinity tag; and
c) separating the target protein from the first composition.

22. The method of claim 21, wherein the cleavage agent comprises a metal ion.

23. The method of claim 22, wherein the cleavage agent comprises copper.

24. The method of claim 22, wherein the cleavage agent comprises Cu(II).

25. The method of claim 22, wherein the cleavage agent partially chelates the metal ion.

26. The method of claim 21, wherein the linker comprises a peptide sequence comprising a tripeptide with the sequence XC₁C₂, wherein X is any amino acid and wherein C₁ is chosen from a cysteine and a cysteine-like nonnatural amino acid, and C₂ is chosen from a cysteine, a cysteine-like nonnatural amino acid, a histidine, and a histidine-like nonnatural amino acid.

27. The method of claim 21, wherein the affinity tag comprises a tag from the group consisting of maltose binding protein, gluotathione-S-transferase, poly(His), biotin ligase tags, Strep, HaloTag, cellulose binding domain, gluotathione transferase, and glycan.

28. The method of claim 21, wherein the ligand is associated with a substrate.

29. The method of claim 21, wherein the affinity tag in said first composition binds the ligand in said affinity material irreversibly.

30. The method of claim 21, wherein the linker in said first composition binds to and removes the metal ion from said cleavage agent.

31. The method of claim 21, wherein the method further comprises preparing the target protein for therapeutic administration.

32. The method of claim 21, wherein the affinity material is discarded after a single use.

33. A composition for purifying a protein comprising;
a) an affinity tag, wherein the affinity tag is capable of binding to a ligand; and
b) a linker associated with the affinity tag, wherein the linker is covalently attached to a target protein;
wherein the entirety of the affinity tag and the linker are capable of being detached from the target protein by the addition of a cleavage agent comprising a metal ion.

34. (canceled)

35. (canceled)

36. The composition of claim 33, wherein the linker comprises a peptide sequence comprising a tripeptide with the sequence XC₁C₂, wherein X is any amino acid and wherein C₁ is chosen from a cysteine and a cysteine-like nonnatural amino acid, and C₂ is chosen from a cysteine, a cysteine-like nonnatural amino acid, a histidine, and a histidine-like nonnatural amino acid.

37. The composition of claim 33, wherein the cleavage agent comprises copper.

38. The composition of claim 37, wherein the cleavage agent comprises Cu(II).

39. The composition of claim 33, wherein the metal ion is partially chelated in the cleavage agent.

40. The composition of claim 33, wherein the affinity tag comprises a tag from the group consisting of maltose binding protein, gluotathione-S-transferase, poly(His), biotin ligase tags, Strep, HaloTag, cellulose binding domain, gluotathione transferase, and glycan.

41. The composition of claim 33, wherein the ligand is associated with a substrate.

42. The composition of claim 33, wherein the affinity tag is capable of binding the ligand irreversibly.

43. The composition of claim 33, wherein the linker is capable of capturing the metal ion in said cleavage agent irreversibly.

44. A protein purified according to the method of claim 21.

45. A composition comprising a protein according to claim 44 and further comprising said protein coupled with (i) an affinity tag, (ii) a linker, or (iii) an affinity tag and a linker.

46. The composition of claim 45, wherein said linker comprises a peptide sequence comprising a tripeptide with the sequence XC₁C₂, wherein X is any amino acid and wherein C₁ is chosen from a cysteine and a cysteine-like nonnatural amino acid, and C₂ is chosen from a cysteine, a cysteine-like nonnatural amino acid, a histidine, and a histidine-like nonnatural amino acid.

47. A composition comprising a protein according to claim 44 and further comprising a cleavage agent wherein said cleavage agent comprises a partial chelator and a metal.

48. The composition of claim 47, wherein said metal is copper.


SEQUENCE LISTING (44 sequences)

SEQ ID NO: 1 - protein, 7 aa, Homo sapiens
  MISC_FEATURE (2)..(2): Xaa is any amino acid
NXEQVSP

SEQ ID NO: 2 - DNA, 486 bp, Unknown
  Description of Unknown Cellulose binding domain polynucleotide
gcagcgacatcatcaatgtcagttgaattttacaactctaacaaatcagcacaaacaaactcaattacaccaataatcaaaattactaacacatctgacagtgatttaaatttaaatgacgtaaaagttagatattattacacaagtgatggtacacaaggacaaactttctggtgtgaccatgctggtgcattattaggaaatagctatgttgataacactagcaaagtgacagcaaacttcgttaaagaaacagcaagcccaacatcaacctatgatacatatgttgaatttggatttgcaagcggagcagctactcttaaaaaaggacaatttataactattcaaggaagaataacaaaatcagactggtcaaactacactcaaacaaatgactattcatttgatgcaagtagttcaacaccagttgtaaatccaaaagttacaggatatataggtggagctaaagtacttggtacagcacca

SEQ ID NO: 3 - DNA, 486 bp, Unknown
  Description of Unknown Cellulose binding domain polynucleotide
tggtgctgtaccaagtactttagctccacctatatatcctgtaacttttggatttacaactggtgttgaactacttgcatcaaatgaatagtcatttgtttgagtgtagtttgaccagtctgattttgttattcttccttgaatagttataaattgtccttttttaagagtagctgctccgcttgcaaatccaaattcaacatatgtatcataggttgatgttgggcttgctgtttctttaacgaagtttgctgtcactttgctagtgttatcaacatagctatttcctaataatgcaccagcatggtcacaccagaaagtttgtccttgtgtaccatcacttgtgtaataatatctaacttttacgtcatttaaatttaaatcactgtcagatgtgttagtaattttgattattggtgtaattgagtttgtttgtgctgatttgttagagttgtaaaattcaactgacattgatgatgtcgctgc

SEQ ID NO: 4 - protein, 162 aa, Unknown
  Description of Unknown Cellulose binding domain polypeptide
AATSSMSVEFYNSNKSAQTNSITPIIKITNTSDSDLNLNDVKVRYYYTSDGTQGQTFWCDHAGALLGNSYVDNTSKVTANFVKETASPTSTYDTYVEFGFASGAATLKKGQFITIQGRITKSDWSNYTQTNDYSFDASSSTPVVNPKVTGYIGGAKVLGTAP

SEQ ID NO: 5 - protein, 109 aa, Cellulomonas fimi
APGCRVDYAVTNQWPGGFGANVTITNLGDPVSSWKLDWTYTAGQRIQQLWNGTASTNGGQVSVTSLPWNGSIPTGGTASFGFNGSWAGSNPTPASFSLNGTTCTGTVPT

SEQ ID NO: 6 - protein, 108 aa, Cellulomonas fimi
SGPAGCQVLWGVNQWNTGFTANVTVKNTSSAPVDGWTLTFSFPSGQQVTQAWSSTVTQSGSAVTVRNAPWNGSIPAGGTAQFGFNGSHTGTNAAPTAFSLNGTPCTVG

SEQ ID NO: 7 - protein, 104 aa, Microbispora bispora
QPPAGRACEATYALVNQWPGGFQAEVTVKNTGSSPINGWTVQWTLPSGQSITQLWNGDLSTSGSNVTVRNVSWNGNVPAGGSTSFGFLGSGTGQLSSSITCSAS

SEQ ID NO: 8 - protein, 106 aa, Cellulomonas flavigena
TGSCKVEYNASSWNTGFTASVRVTNTGTTALNGWTLTFPFANGQTVQQGWSADWSQSGTTVTAKNAAWNGSLAAGQTVDIGFNGAHNGTNNKPASFTLNGATCTVG

SEQ ID NO: 9 - protein, 103 aa, Pseudomonas fluorescens
AASGGNCQYVVTNQWNNGFTAVIRVRNNGSSAINRWSVNWSYSDGSRITNSWNANVTGNNPYAASALGWNANIQPGQTAEFGFQGTKGAGSRQVPAVTGSVCQ

SEQ ID NO: 10 - protein, 104 aa, Pseudomonas fluorescens
QTATCSYNITNEWNTGYTGDITITNRGSSAINGWSVNWQYATNRLSSSWNANVSGSNPYSASNLSWNGNIQPGQSVSFGFQVNKNGGSAERPSVGGSICSGSVA

SEQ ID NO: 11 - protein, 97 aa, Butyivibria fibrisolvens
VSGALKAEYTINNWGSGYQVLIKVKNDSASRVDGWTLKISKSEVKIDSSWCVNIAEEGGYYVITPMSWNSSLEPSASVDFGIQGSGSIGTSVNISVQ

SEQ ID NO: 12 - DNA, 158 bp, Artificial Sequence
  Description of Artificial Sequence Synthetic polynucleotide
cttccatggctacccagtctcactacggccagtgcggcggtattgcctacagcggccccacggtctgcgccagcggcacaacttgccaggtcctgaacccttactactctcagtgcctgccaactactccaactggtcgtggtgacagcgctagctga

SEQ ID NO: 13 - protein, 50 aa, Artificial Sequence
  Description of Artificial Sequence Synthetic polypeptide
MATQSHYGQCGGIGYSGPTVCASGTTCQVLNPYYSQCLPTTPTGRGDSAS

SEQ ID NO: 14 - protein, 36 aa, Trichoderma konigii
TQSHYGQCGGIGYSGPTVCASGTTCQVLNPYYSQCL

SEQ ID NO: 15 - DNA, 108 bp, Trichoderma konigii
  CDS (1)..(108)
acccagtctcactacggccagtgcggcggtattgcctacagcggccccacggtctgcgccagcggcacaacttgccaggtcctgaacccttactactctcagtgcctg

SEQ ID NO: 16 - protein, 36 aa, Trichoderma konigii; identical to SEQ ID NO: 14

SEQ ID NO: 17 - protein, 44 aa, Artificial Sequence
  Description of Artificial Sequence Synthetic polypeptide
MATQSHWGQCGGIGYSGPTVCASGTTCQVLNPYYSQCLPTTPTG

SEQ ID NO: 18 - protein, 209 aa, Artificial Sequence
  Description of Artificial Sequence Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLLANLAGSGSGHMHHHHHHSSGLVPRGSGMLETAAAKFERQHMDSPDLGTDDDDKAMATQSHWGQCGGIGYSGPTVCASGTTCQVLNPYYSQCLPTTPTGRGDSAS

SEQ ID NO: 19 - protein, 36 aa, Trichoderma konigii; identical to SEQ ID NO: 14

SEQ ID NO: 20 - DNA, 161 bp, Artificial Sequence
  Description of Artificial Sequence Synthetic polynucleotide
cttccatggctacccagtctcactggggccagtgcggcggtattgcctacagcggcccccccacggtctgcgccagcggcacaacttgccaggtcctgaacccttactactctcagtgcctgccaactactccaactggtcgtggtgacagcgctagctga

SEQ ID NO: 21 - DNA, 161 bp, Artificial Sequence
  Description of Artificial Sequence Synthetic polynucleotide
tcagctagcgctgtcaccacgaccagttggagtagttggcaggcactgagagtagtaagggttcaggacctggcaagttgtgccgctggcgcagaccgtgggggggccgctgtaggcaataccgccgcactggccccagtgagactgggtagccatggaag

SEQ ID NO: 22 - protein, 51 aa, Artificial Sequence
  Description of Artificial Sequence Synthetic polypeptide
MATQSHWGQCGGIGYSGPPTVCASGTTCQVLNPYYSQCLPTTPTGRGDSAS

SEQ ID NO: 23 - DNA, 35 bp, Artificial Sequence
  Description of Artificial Sequence Synthetic primer
ggtcatagaaacaactcttgcattcaataaggatc

SEQ ID NO: 24 - DNA, 41 bp, Artificial Sequence
  Description of Artificial Sequence Synthetic primer
ggtcatagaaacaactgttccattcaataaggctgtaactc

SEQ ID NO: 25 - DNA, 43 bp, Artificial Sequence
  Description of Artificial Sequence Synthetic primer
cgtttcaaacattccaacggtgctagaaacaactgttgcattc

SEQ ID NO: 26 - protein, 5 aa, Artificial Sequence
  Description of Artificial Sequence Synthetic peptide
GGNCC

SEQ ID NO: 27 - protein, 5 aa, Artificial Sequence
  Description of Artificial Sequence Synthetic peptide
GGGCC

SEQ ID NO: 28 - protein, 5 aa, Artificial Sequence
  Description of Artificial Sequence Synthetic peptide
GNNCC

SEQ ID NO: 29 - protein, 5 aa, Artificial Sequence
  Description of Artificial Sequence Synthetic peptide
GNGCC

SEQ ID NO: 30 - protein, 8 aa, Artificial Sequence
  Description of Artificial Sequence Synthetic peptide
GGNCCGGK

SEQ ID NO: 31 - protein, 8 aa, Artificial Sequence
  Description of Artificial Sequence Synthetic peptide
GGGCCGGK

SEQ ID NO: 32 - protein, 8 aa, Artificial Sequence
  Description of Artificial Sequence Synthetic peptide
GGNCHGGK

SEQ ID NO: 33 - protein, 6 aa, Artificial Sequence
  Description of Artificial Sequence Synthetic peptide
NCCGGK

SEQ ID NO: 34 - protein, 5 aa, Artificial Sequence
  Description of Artificial Sequence Synthetic peptide
CCGGK

SEQ ID NO: 35 - protein, 4 aa, Artificial Sequence
  Description of Artificial Sequence Synthetic peptide
CGGK

SEQ ID NO: 36 - protein, 4 aa, Artificial Sequence
  Description of Artificial Sequence Synthetic peptide
GGGC

SEQ ID NO: 37 - protein, 6 aa, Artificial Sequence
  Description of Artificial Sequence Synthetic peptide
GCCGGK

SEQ ID NO: 38 - protein, 5 aa, Artificial Sequence
  Description of Artificial Sequence Synthetic peptide
CCGGK

SEQ ID NO: 39 - protein, 4 aa, Artificial Sequence
  Description of Artificial Sequence Synthetic peptide
CGGK

SEQ ID NO: 40 - protein, 23 aa, Homo sapiens
KYRPKMRLRFKDSNGHRNNCCIQ

SEQ ID NO: 41 - protein, 20 aa, Homo sapiens
KYRPKMRLRFRDTNGHCCVQ

SEQ ID NO: 42 - protein, 23 aa, Homo sapiens
KYRPKQRLRFKDPHTHKTRCCVM

SEQ ID NO: 43 - protein, 6 aa, Artificial Sequence
  Description of Artificial Sequence Synthetic 6xHis tag
HHHHHH

SEQ ID NO: 44 - protein, 4 aa, Artificial Sequence
  Description of Artificial Sequence Synthetic peptide
GGNC